SEQUENCE LISTING (90 sequences)

SEQ ID NO: 1 - protein, 7 aa, synthetic construct
VNMHTRP

SEQ ID NO: 2 - protein, 7 aa, synthetic construct
HATGSPR

SEQ ID NO: 3 - protein, 7 aa, synthetic construct
VLTATRP

SEQ ID NO: 4 - protein, 7 aa, synthetic construct
VITPTRP

SEQ ID NO: 5 - protein, 7 aa, synthetic construct
VNEPRRP

SEQ ID NO: 6 - protein, 7 aa, synthetic construct
VNNKTRP

SEQ ID NO: 7 - protein, 7 aa, synthetic construct
WPPKTTS

SEQ ID NO: 8 - protein, 7 aa, synthetic construct
PTSKTTS

SEQ ID NO: 9 - protein, 7 aa, synthetic construct
LMVKTTS

SEQ ID NO: 10 - protein, 7 aa, synthetic construct
WTDKTTS

SEQ ID NO: 11 - protein, 7 aa, synthetic construct
QRPPREP

SEQ ID NO: 12 - protein, 7 aa, synthetic construct
TAFPREP

SEQ ID NO: 13 - protein, 11 aa, synthetic construct
AQWPPKTTSAQ

SEQ ID NO: 14 - protein, 11 aa, synthetic construct
AQPTSKTTSAQ

SEQ ID NO: 15 - protein, 11 aa, synthetic construct
AQLMVKTTSAQ

SEQ ID NO: 16 - protein, 11 aa, synthetic construct
AQWTDKTTSAQ

SEQ ID NO: 17 - protein, 11 aa, synthetic construct
AQQRPPREPAQ

SEQ ID NO: 18 - protein, 11 aa, synthetic construct
AQTAFPREPAQ

SEQ ID NO: 19 - protein, 11 aa, synthetic construct
LMTPPKTTSAQ

SEQ ID NO: 20 - protein, 11 aa, synthetic construct
ATEPPKTTSAQ

SEQ ID NO: 21 - protein, 11 aa, synthetic construct
AVLSPKTTSAQ

SEQ ID NO: 22 - protein, 11 aa, synthetic construct
AQQRPPRPADQ

SEQ ID NO: 23 - protein, 11 aa, synthetic construct
APARPPREPAQ

SEQ ID NO: 24 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 25 - protein, 743 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPVNMHTRPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 26 - protein, 743 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPHATGSPRATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 27 - protein, 743 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPVLTATRPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 28 - protein, 743 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPVITPTRPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 29 - protein, 743 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPVNEPRRPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 30 - protein, 743 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPVNNKTRPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 31 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 32 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQWPPKTTSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 33 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQQRPPREPAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 34 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQQRPPRPADQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 35 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSLMTPPKTTSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 36 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSATEPPKTTSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 37 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTAFPREPAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 38 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAPARPPREPAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 39 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQPTSKTTSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 40 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQLMVKTTSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 41 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQWTDKTTSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 42 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAVLSPKTTSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 43 - protein, 45 aa, synthetic construct
TTTPAPRPPTPAPTIASQPLSLRPEACRPAAGGAVHTRGLDFACD

SEQ ID NO: 44 - protein, 24 aa, synthetic construct
IYIWAPLAGTCGVLLLSLVITLYC

SEQ ID NO: 45 - protein, 42 aa, synthetic construct
KRGRKKLLYIFKQPFMRPVQTTQEEDGCSCRFPEEEEGGCEL

SEQ ID NO: 46 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 223 aa, synthetic construct
TTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - DNA, 36 bp, synthetic construct
  source 1..36: mol_type = other DNA
actcatcgaccaatacttgtactatctctctagaac

SEQ ID NO: 49 - DNA, 83 bp, synthetic construct
  source 1..83: mol_type = other DNA
gtattccttggttttgaacccaaccggtctgcgcctgtgcmnnmnnmnnmnnmnnmnnmnnttgggcactctggtggtttgtg

SEQ ID NO: 50 - DNA, 27 bp, synthetic construct
  source 1..27: mol_type = other DNA
caggtcttcacggactcagactatcag

SEQ ID NO: 51 - DNA, 34 bp, synthetic construct
  source 1..34: mol_type = other DNA
cattgatgagtttggacaaaccacaactagaatg

SEQ ID NO: 52 - DNA, 56 bp, synthetic construct
  source 1..56: mol_type = other DNA
acactctttccctacacgacgctcttccgatctgacaagtggccacaaaccaccag

SEQ ID NO: 53 - DNA, 55 bp, synthetic construct
  source 1..55: mol_type = other DNA
gtgactggagttcagacgtgtgctcttccgatctccttggttttgaacccaaccg

SEQ ID NO: 54 - DNA, 34 bp, synthetic construct
  source 1..34: mol_type = other DNA
cattctagttgtggtttgtccaaactcatcaatg

SEQ ID NO: 55 - DNA, 25 bp, synthetic construct
  source 1..25: mol_type = other DNA
aatagcttgatgaatcctggacctg

SEQ ID NO: 56 - DNA, 23 bp, synthetic construct
  source 1..23: mol_type = other DNA
ttccttggttttgaacccaaccg

SEQ ID NO: 57 - DNA, 83 bp, synthetic construct
  source 1..83: mol_type = other DNA
gtattccttggttttgaacccaaccggtctgcgcctgtgcagacgtagtcttcggcggmnnmnnmnnactctggtggtttgtg

SEQ ID NO: 58 - DNA, 83 bp, synthetic construct
  source 1..83: mol_type = other DNA
gtattccttggttttgaacccaaccggtctgcgcctgtgcagacgtagtcttcggmnnmnnmnnggcactctggtggtttgtg

SEQ ID NO: 59 - DNA, 83 bp, synthetic construct
  source 1..83: mol_type = other DNA
gtattccttggttttgaacccaaccggtctgcgcctgtgcagacgtagtcttmnnmnnmnnttgggcactctggtggtttgtg

SEQ ID NO: 60 - DNA, 83 bp, synthetic construct
  source 1..83: mol_type = other DNA
gtattccttggttttgaacccaaccggtctgcgcctgtgcagacgtagtmnnmnnmnnccattgggcactctggtggtttgtg

SEQ ID NO: 61 - DNA, 83 bp, synthetic construct
  source 1..83: mol_type = other DNA
gtattccttggttttgaacccaaccggtctgcgcctgtgcagacgtmnnmnnmnncggccattgggcactctggtggtttgtg

SEQ ID NO: 62 - DNA, 83 bp, synthetic construct
  source 1..83: mol_type = other DNA
gtattccttggttttgaacccaaccggtctgcgcctgtgcagamnnmnnmnncggcggccattgggcactctggtggtttgtg

SEQ ID NO: 63 - DNA, 83 bp, synthetic construct
  source 1..83: mol_type = other DNA
gtattccttggttttgaacccaaccggtctgcgcctgtgcmnnmnnmnncttcggcggccattgggcactctggtggtttgtg

SEQ ID NO: 64 - DNA, 83 bp, synthetic construct
  source 1..83: mol_type = other DNA
gtattccttggttttgaacccaaccggtctgcgcctgmnnmnnmnnagtcttcggcggccattgggcactctggtggtttgtg

SEQ ID NO: 65 - DNA, 83 bp, synthetic construct
  source 1..83: mol_type = other DNA
gtattccttggttttgaacccaaccggtctgcgcmnnmnnmnncgtagtcttcggcggccattgggcactctggtggtttgtg

SEQ ID NO: 66 - DNA, 83 bp, synthetic construct
  source 1..83: mol_type = other DNA
gtattccttggttttgaacccaaccggtctgcgcmnnmnnagacgtagtcttcggcggccamnnmnnactctggtggtttgtg

SEQ ID NO: 67 - DNA, 83 bp, synthetic construct
  source 1..83: mol_type = other DNA
gtattccttggttttgaacccaaccggtctgcgcctgtgccggctcacgcggaggcctmnnmnnmnnactctggtggtttgtg

SEQ ID NO: 68 - DNA, 83 bp, synthetic construct
  source 1..83: mol_type = other DNA
gtattccttggttttgaacccaaccggtctgcgcctgtgccggctcacgcggaggmnnmnnmnnggcactctggtggtttgtg

SEQ ID NO: 69 - DNA, 83 bp, synthetic construct
  source 1..83: mol_type = other DNA
gtattccttggttttgaacccaaccggtctgcgcctgtgccggctcacgcggmnnmnnmnnttgggcactctggtggtttgtg

SEQ ID NO: 70 - DNA, 83 bp, synthetic construct
  source 1..83: mol_type = other DNA
gtattccttggttttgaacccaaccggtctgcgcctgtgccggctcacgmnnmnnmnnctgttgggcactctggtggtttgtg

SEQ ID NO: 71 - DNA, 83 bp, synthetic construct
  source 1..83: mol_type = other DNA
gtattccttggttttgaacccaaccggtctgcgcctgtgccggctcmnnmnnmnncctctgttgggcactctggtggtttgtg

SEQ ID NO: 72 - DNA, 83 bp, synthetic construct
  source 1..83: mol_type = other DNA
gtattccttggttttgaacccaaccggtctgcgcctgtgccggmnnmnnmnnaggcctctgttgggcactctggtggtttgtg

SEQ ID NO: 73 - DNA, 83 bp, synthetic construct
  source 1..83: mol_type = other DNA
gtattccttggttttgaacccaaccggtctgcgcctgtgcmnnmnnmnncggaggcctctgttgggcactctggtggtttgtg

SEQ ID NO: 74 - DNA, 83 bp, synthetic construct
  source 1..83: mol_type = other DNA
gtattccttggttttgaacccaaccggtctgcgcctgmnnmnnmnnacgcggaggcctctgttgggcactctggtggtttgtg

SEQ ID NO: 75 - DNA, 83 bp, synthetic construct
  source 1..83: mol_type = other DNA
gtattccttggttttgaacccaaccggtctgcgcmnnmnnmnnctcacgcggaggcctctgttgggcactctggtggtttgtg

SEQ ID NO: 76 - DNA, 83 bp, synthetic construct
  source 1..83: mol_type = other DNA
gtattccttggttttgaacccaaccggtctgcgcmnnmnncggctcacgcggaggcctctgmnnmnnactctggtggtttgtg

SEQ ID NO: 77 - DNA, 26 bp, synthetic construct
  source 1..26: mol_type = other DNA
agaactcaaaatcagtccggaagtgc

SEQ ID NO: 78 - DNA, 62 bp, synthetic construct
  source 1..62: mol_type = other DNA
gcatgcacatctccggtcgcmnnmnnmnnmnnmnnmnnmnnagggtctgtgctgctgctctg

SEQ ID NO: 79 - DNA, 30 bp, synthetic construct
  source 1..30: mol_type = other DNA
ccttttactgcctggaatatttcccttctc

SEQ ID NO: 80 - DNA, 20 bp, synthetic construct
  source 1..20: mol_type = other DNA
gcatgcacatctccggtcgc

SEQ ID NO: 81 - DNA, 69 bp, synthetic construct
  source 1..69: mol_type = other DNA
gcatgcacatctccggtcgcaggcctagtatgcatattmnnmnnmnntgtgctgctgctctggaaattg

SEQ ID NO: 82 - DNA, 69 bp, synthetic construct
  source 1..69: mol_type = other DNA
gcatgcacatctccggtcgcaggcctagtatgcatmnnmnnmnngtctgtgctgctgctctggaaattg

SEQ ID NO: 83 - DNA, 69 bp, synthetic construct
  source 1..69: mol_type = other DNA
gcatgcacatctccggtcgcaggcctagtatgmnnmnnmnnagggtctgtgctgctgctctggaaattg

SEQ ID NO: 84 - DNA, 69 bp, synthetic construct
  source 1..69: mol_type = other DNA
gcatgcacatctccggtcgcaggcctagtmnnmnnmnnaacagggtctgtgctgctgctctggaaattg

SEQ ID NO: 85 - DNA, 69 bp, synthetic construct
  source 1..69: mol_type = other DNA
gcatgcacatctccggtcgcaggcctmnnmnnmnnattaacagggtctgtgctgctgctctggaaattg

SEQ ID NO: 86 - DNA, 69 bp, synthetic construct
  source 1..69: mol_type = other DNA
gcatgcacatctccggtcgcaggmnnmnnmnncatattaacagggtctgtgctgctgctctggaaattg

SEQ ID NO: 87 - DNA, 69 bp, synthetic construct
  source 1..69: mol_type = other DNA
gcatgcacatctccggtcgcmnnmnnmnnatgcatattaacagggtctgtgctgctgctctggaaattg

SEQ ID NO: 88 - DNA, 69 bp, synthetic construct
  source 1..69: mol_type = other DNA
gcatgcacatctccggtmnnmnnmnnagtatgcatattaacagggtctgtgctgctgctctggaaattg

SEQ ID NO: 89 - DNA, 69 bp, synthetic construct
  source 1..69: mol_type = other DNA
gcatgcacatctccmnnmnnmnncctagtatgcatattaacagggtctgtgctgctgctctggaaattg

SEQ ID NO: 90 - DNA, 69 bp, synthetic construct
  source 1..69: mol_type = other DNA
gcatgcacatctccmnnmnnaggcctagtatgcatattaacmnnmnntgtgctgctgctctggaaattg

(19) United States
(12) Patent Application Publication
(10) Pub. No.: US 2025/0333758 A1
(43) Pub. Date: Oct. 30, 2025

(54) MICROGLIA HAVING CAR AND USE THEREOF

(71) Applicant: GENANS BIOTECHNOLOGY CO., LTD, Beijing (CN)

(72) Inventors: Rui Lin, Beijing (CN); Ting Yan, Beijing (CN); Minmin Luo, Beijing (CN)

(21) Appl. No.: 18/720,949

(22) PCT Filed: Dec. 15, 2022
(86) PCT No.: PCT/CN2022/139356

§ 371 Date: Jun. 17, 2024

(30) Foreign Application Priority Data
Dec. 15, 2021 (WO) PCT/CN2021/138535
May 31, 2022 (WO) PCT/CN2022/096277
Jun. 21, 2022 (WO) PCT/CN2022/100166

(51) Int. Cl. C12N 15/86; A61K 35/30; A61K 40/10; A61K 40/31; A61K 40/42; A61K 48/00; A61P 35/00; C07K 14/005; C07K 14/705; C12N 5/079

(52) U.S. Cl. CPC C12N 15/86; A61K 35/30; A61K 40/10; A61K 40/31; A61K 40/428; A61K 48/0008; A61P 35/00; C07K 14/005; C07K 14/70596; C12N 5/0622; A61K 2239/17; A61K 2239/21; A61K 2239/22; C12N 2510/00; C12N 2750/14143

(57) ABSTRACT

Provided is a recombinant adeno-associated virus (rAAV) vector comprising a nucleic acid molecule encoding a chimeric antigen receptor (CAR) which specifically binds to a central nervous system (CNS) tumor cell, preferably a solid CNS tumor cell. Further provided is a modified cell comprising a chimeric antigen receptor (CAR) which is obtained by transducing the cell with the rAAV vector, and a method for treating a CNS tumor using the rAAV vector or modified cell of the present disclosure.

### FIELD OF THE INVENTION

The present disclosure relates to the field of biological medicine, in particular to microglia having a chimeric antigen receptor (CAR) and use thereof.

### REFERENCE TO SEQUENCE LISTING

The sequence listing submitted on Jan. 22, 2025, as an .XML file entitled “WO11437BSUS-Sequence listing.xml” created on Jan. 20, 2025, and having a file size of 97,034 bytes is hereby incorporated by reference pursuant to 37 C.F.R. § 1.52 (e) (5).

### BACKGROUND

In recent years, chimeric antigen receptor T (CAR-T) cell therapy has developed rapidly. CAR-T cell therapy is a way to get immune cells (e.g. T cells) to fight cancer by changing the immune cells so that they can find and destroy cancer cells. For the CAR-T cell therapy, T cells are collected from a patient, engineered to express CAR, and then infused into the patient after multiplication. The engineered CAR-T cell can recognize and attack cells that have the targeted antigen on their surface.

There are still many obstacles to the application of CAR-T therapy to the treatment of solid tumors. For example, the autologous CAR-T cells have low survivability and multiplication capacity in patients with malignant solid tumors. At the same time, the tumor microenvironment (TME) can actively recruit myeloid cells, leading to extensive infiltration with immunosuppressive macrophages which constitute tumor-associated macrophages (TAMs). TAMs have weak phagocytosis and lack binding specificity for tumor-associated antigens. However, TAMs still can release a variety of growth factors and cytokines in response to factors released by tumor cells, thereby promoting tumor survival, proliferation and migration.

Central nervous system (CNS) tumor is an abnormal growth of cells from the tissues of the brain or spinal cord. The CNS tumor contains a large number of TAMs that originate from peripheral or brain microglia. Microglia are the only resident myeloid cells in the central nervous system, and have functions similar to that of peripheral macrophages. To date, it is difficult to transfect microglia with those vectors conventionally used in gene therapy, such as recombinant adeno-associated virus (rAAV). So, the effect of CAR-T therapy in the treatment of glioma is not ideal.

Therefore, there is an urgent need for effective targeted therapies of CNS tumors.

### SUMMARY OF THE INVENTION

To overcome at least one of the above technical problems, the present disclosure provides potential new strategies for treating tumors of central nervous system (CNS).

According to one aspect, provided is a recombinant adeno-associated virus (rAAV) vector, comprising a nucleic acid molecule encoding a chimeric antigen receptor (CAR) which specifically binds to a central nervous system (CNS) tumor cell. According to some embodiments, the CAR can specifically bind to a solid CNS tumor cell.

According to some embodiments, the rAAV vector comprises a capsid protein, which has an inserted amino acid sequence of seven contiguous amino acids in a GH-loop of the capsid protein. According to some embodiments, the capsid protein comprises an amino acid sequence selected from a group consisting of SEQ ID NO: 1, SEQ ID NO: 2, SEQ ID NO: 3, SEQ ID NO: 4, SEQ ID NO: 5 and SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 19, SEQ ID NO: 20, SEQ ID NO: 21, SEQ ID NO: 22, and SEQ ID NO: 23.

According to another aspect, provided is a modified cell, preferably a modified microglia and/or astrocyte, which comprises a chimeric antigen receptor (CAR) which specifically binds to a central nervous system (CNS) tumor cell, such as a solid CNS tumor cell.

According to yet another aspect, provided is a pharmaceutical composition which comprises the above rAAV vector or the above modified cell.

According to yet another aspect, provided is a method for treating a CNS tumor, preferably a solid CNS tumor, comprising administering to a subject a therapeutically effective amount of the above rAAV vector, the above modified cell, or the above pharmaceutical composition.

According to yet another aspect, provided is use of the above rAAV vector, the above modified cell, or the above pharmaceutical composition for treating a CNS tumor, preferably a solid CNS tumor.

According to yet another aspect, provided is use of the above rAAV vector or the above modified cell in the manufacture of a composition for treating a CNS tumor, preferably a solid CNS tumor.

The present disclosure obtained rAAV vectors with high transduction rate for microglia. With such rAAV vectors, microglia can be modified and introduced with CAR which specifically bind to CNS tumor cells. The modified microglia can be activated by CNS tumor cells, to release proinflammatory cytokines such as IL6, I11β, Nos2 and TNF-α. Further, the modified microglia expressing CAR can specifically recognize and phagocytose CNS tumor cells. Once transplanted into the brain, the modified microglia can locate correctly, and then recognize and destroy tumor cells.

### DESCRIPTION OF THE FIGURES

FIG. 1. Screen of AAV9-MGs that mediate efficient microglial transduction. (A) Schematic diagram of the in vitro screening process in which random heptamers were inserted between the 588 and 589 amino acids of the AAV9 VP1 protein. The library was screened in cultured mouse microglia for two rounds. (B) Distributions of AAV9 capsid variants recovered from cultured mouse microglia, sorted by decreasing order of the enrichment score. The pie chart shows the normalized frequency of AAV-cMG.WPP among total recovered sequences. (C) Representative images of cultured mouse microglia transduced with mScarlet reporter rAAVs packaged using different capsids. (D) Quantification of the mScarlet⁺ percentage and the mean fluorescent intensity of cultured mouse microglia transduced with mScarlet reporter rAAVs packaged using different capsids (n=4 replicates for each group; the bar represents the mean value for each group; one-way ANOVA with Dunnett's post-hoc test).

FIG. 2. Screen of AAV-cMG.QRP that mediate efficient microglial transduction. (A) Distributions of AAV9 capsid variants recovered from cultured mouse microglia, sorted by decreasing order of the enrichment score. The pie chart shows the normalized frequency of AAV-cMG.QRP in total recovered sequences. (B) Representative images of cultured mouse microglia transduced with mScarlet reporter AAVs packaged using different capsids. (C) Quantification of mScarlet⁺ percentage and the mean fluorescent intensity of cultured mouse microglia transduced with mScarlet reporter AAVs packaged using different capsids (n=6 replicates for each group in 3 pt; 5 replicates for each group in 5 pt; the bar represents the mean value for each group; one-way ANOVA with Dunnett's post-hoc test).

FIG. 3. In vivo screen of AAV-cMG.WPP variants that mediate efficient microglial transduction. (A) Distributions of AAV-cMG.WPP variants recovered from the Cx3cr1^{CreER} mouse brains, sorted by decreasing order of the enrichment score. The pie chart shows the normalized frequency of AAV-MG1.1 and AAV-MG1.2 among total recovered sequences. Magenta: AAV-MG1.1, green: AAV-MG1.2, cyan: AAV-cMG.WPP. (B-E) Representative images showing the mScarlet expression patterns in the striatum of Cx3cr1^{CreER} mice injected with (B) AAV-MG. PTS-SFFV-DIO-mScarlet, (C) AAV-MG.LMV-SFFV-DIO-mScarlet, (D) AAV-MG.WTD-SFFV-DIO-mScarlet, or (E) AAV-MG.VLS-SFFV-DIO-mScarlet. Scale bars, 500 μm.

FIG. 4. In vivo screen of AAV-MG.QRP variants that mediate efficient microglial transduction. (A) Schematic of the selection process of AAV-MG.QRP variants. The right panel shows distributions of AAV-MG.QRP variants recovered from cultured mouse microglia, sorted by decreasing order of the enrichment score. The pie chart shows the normalized frequency of AAV-cMG in total recovered sequences. (B-C) Representative images showing the mScarlet expression patterns in the striatum of Cx3cr1^{CreER} mice injected with (B) AAV-MG.TAF-SFFV-DIO-mScarlet or (C) AAV-MG.APA-SFFV-DIO-mScarlet.

FIG. 5. Directed evolution of AAV1 capsid generates AAV-cMG variants mediating efficient gene transduction in cultured microglia. (A) Schematic of the selection process. Random seven amino acids were inserted between the 591 and 592 amino acids of the AAV1 VP1 protein. The library was screened in cultured mouse microglia for two rounds. (B) Distributions of AAV1 capsid variants recovered from cultured mouse microglia, sorted by decreasing order of the enrichment score. The pie chart shows the normalized frequency of AAV-cMG.HAT (2.96%) and AAV-cMG.VNM (0.57%) in total recovered sequences. (C) Schematic of the selection process of AAV-cMG.VNM variants. The right panel shows distributions of AAV-cMG.VNM variants recovered from cultured mouse microglia, sorted by decreasing order of the enrichment score. The pie chart shows the normalized frequency of AAV-cMG1.1 (0.34%) and AAV-cMG1.2 (0.37%) in total recovered sequences.

FIG. 6. AAV-cMG2 mediates efficient gene transduction in cultured microglia. (A) Representative images of cultured mouse microglia transduced with mScarlet reporter AAVs packaged using different capsids. Scale bar, 200 μm. (B) Quantification of the mean fluorescent intensity and mScarlet+ percentage of cultured mouse microglia transduced with mScarlet reporter AAVs packaged using different capsids (n=3; MOI: 105; 5 days post-transduction; one-way ANOVA with Dunnett's post-hoc test; P values as listed in the figure). Data are presented as scatter and mean.

FIG. 7. AAV-cMG2 shows higher AAV packaging yields compared with AAV-cMG. Quantification of the titer of AAVs packaged using different capsids (n=3; one-way ANOVA with Dunnett's post-hoc test; P values as listed in the figure). Data are presented as scatter and mean. Typically, rAAVs were packaged by using 5×107 cells in three 15-cm petri dishes and resuspended in 400 μL PBS.

FIG. 8. Doxorubicin enhances AAV-cMG2 microglial transduction. Quantification of the mean fluorescent intensity of cultured mouse microglia transduced with mScarlet reporter AAVs packaged using AAV-cMG or AAV-cMG2 (n=3; MOI: 105; 5 days post-transduction; one-way ANOVA with Dunnett's post-hoc test; P values as listed in the figure). Data are presented as scatter and mean.

FIG. 9. AAV-cMG2 drives strong and functional chimeric antigen receptors (CARs) expression in microglia. (A) Design of the AAV vector expressing the B7H3-CAR. mAb: monoclonal antibody; TM: transmembrane domain; ICD: intracellular domain. (B) Representative immunofluorescence images showing the colocalization of GFP (green) and Myc immunosignals (yellow) in cultured mouse microglia transduced with AAV-cMG2-B7H3-CAR. Scale bar, 200 μm. (C) The binding of B7H3 ECD by B7H3-CAR-Mis in which AAV transduction were performed without doxorubicin. (D) The binding of B7H3 ECD by B7H3-CAR-Mis in which AAV transduction were performed with doxorubicin.

FIG. 10. AAV-cMG2 transduction, CAR expression and doxorubicin treatment do not activate microglia. (A) Principal component analysis of the transcriptomes of cultured mouse microglia from five treatment groups: control untransduced (UTD), lipopolysaccharide (LPS)-treated, interleukin-4-treated (IL4), AAV-cMG2-B7H3-CAR-transduced (CAR-Mi), and doxorubicin-treated AAV-cMG2-B7H3-CAR-transduced (CAR-Mi+Doxo) group (n=3 replicates for each group). (B) Hierarchical clustering performed on marker genes of microglial states for different treatment groups as shown in (A). The color bar represents the z-score of the relative gene expression.

FIG. 11. CAR-Mi cells phagocytose microsphere beads (sp-beads) in a target-specific manner. (A) Representative images showing the colocalization of pHrodo-loaded B7H3 ECDs labeled sp-beads (sp-B7H3-beads) (yellow) and B7H3-CAR-Mi cells (GFP). Scale bar, 25 μm. (B) Quantifications of indicated microglia phagocytosis against sp-beads or sp-B7H3-beads at 0.5 after beads addition. Statistical significance was calculated with one-way ANOVA with multiple comparisons. (C) Time-series analysis of indicated microglia phagocytosis against sp-beads or sp-B7H3-beads. Statistical significance was calculated with one-way ANOVA with multiple comparisons. For all panels, *P<0.05, **P<0.01, ***P<0.001, ****P<0.0001.

FIG. 12. CAR-Mi cells phagocytose live cells in a target-specific manner. (A) Representative images showing the phagocytosis of U87 cells (red) by B7H3-CAR-Mi cells (green). (B) Quantifications of indicated microglia phagocytosis against GL261 cells that stably expressed B7H3 ECDs (GL261-B7H3-ECD). Statistical significance was calculated with one-way ANOVA with multiple comparisons. *P<0.05, **P<0.01, ***P<0.001.

FIG. 13. Secretion of pro-inflammatory cytokines of CAR-Mi cells. Quantifications of IL6 (A) and TNF-α (B) in the culture medium of indicated microglia cultured alone or with GL261-B7H3-ECD cells. Statistical significance was calculated with one-way ANOVA with multiple comparisons. *P<0.05, **P<0.01, ***P<0.001, n.s.>0.05.

FIG. 14. CAR-Mi cells release pro-inflammatory cytokines and activate by-stander microglia upon target cell recognition. Quantifications of pro-inflammatory (IL6, IL1β, TNFα, and Nos2) and anti-inflammatory (Mrc1 and Chi13) marker genes in homeostatic or I14-treated microglia after conditioned with the culture medium of homeostatic microglia (WT MG), CAR-Mi cells, or B7H3-CAR-Mi cells co-cultured with GL261-B7H3-ECD cells (CAR-Mi+GBM). Statistical significance was calculated with one-way ANOVA with multiple comparisons. *P<0.05, **P<0.01, ***P<0.001, n.s.>0.05.

FIG. 15. CAR-Mi cells suppress tumor growth in vivo. (A) Schematic of the experimental procedure. Cx3cr1^{CreER}: Rosa26-LSL-DTA mice were used. GL261-B7H3-ECD cells stably expressed luciferase of imaging. (B) Representative images showing the distribution of transplanted GFP-expressing microglia (green) in the brains of two Cx3cr1^{CreER}: Rosa26-LSL-DTA mice. Scale bar, 25 mm. (C) Quantifications of tumor burden by bioluminescent imaging.

### DETAILED DESCRIPTION OF THE INVENTION

Before the present methods and compositions are described, it is to be understood that this invention is not limited to a particular method or composition described and as such may vary. It is also to be understood that the terminology used herein is for the purpose of describing particular embodiments only, and is not intended to be limiting, since the scope of the present invention will be limited only by the appended claims.

Chimeric antigen receptor T cell (CAR-T) therapy has achieved great success in treating malignant blood cancers, and has been considered as one of the most promising tumor treatment approaches. Applications of CAR-T in solid tumors, however, are challenging due to the inability of T cells to penetrate, as well as the inhibitory tumor microenvironment. The solid tumor microenvironment generates various chemokines that recruit myeloid cells, leading to extensive infiltration of immunosuppressive macrophages known as tumor-associated macrophages (TAMs). TAMs have reduced phagocytosis and lack the capability to bind tumor-associated antigens. Instead, TAMs promote tumor survival, proliferation and migration by releasing a variety of growth factors and cytokines in response to tumor cells. Considering the importance of macrophages in the tumor microenvironment, enormous interests have been sparked to develop therapeutic approaches for depleting or re-activating TAMs.

Microglia are the sole resident immune cells and specialized macrophages in the central nervous system (CNS). Similar to solid tumors in the peripheral system, solid CNS tumors also contain considerable amounts of TAMs which consist of tumor-associated resident microglia and infiltrated peripheral macrophages. For example, in high-grade glioma, non-neoplastic cells are predominantly tumor-associated microglia that are immunosuppressive. The tumor-associated microglia may be engineered for CAR-T therapy for CNS tumors.

However, the transduction rate of microglia was not high enough for using in CAR-T therapy of CNS tumors. For this, in this disclosure, recombinant adeno-associated viruses (rAAVs), that mediate efficient gene delivery to microglia, are provided through screening. Then, these obtained rAAVs are used to deliver CAR molecules into microglia to target CNS tumors. Further, the inventors surprisingly find that CAR-modified microglia can recognize and phagocytose tumor cells, which have great potentials as an approach for treating tumors, especially CNS tumors.

According to one aspect, provided is a recombinant adeno-associated virus (rAAV) vector which comprises a nucleic acid molecule encoding chimeric antigen receptor (CAR) which specifically binds to a CNS tumor cell.

According to some embodiments, the rAAV vector comprises a capsid protein, which has an inserted amino acid sequence of seven contiguous amino acids in a GH-loop of the wide-type capsid protein.

According to some embodiments, the rAAV vector comprises a capsid protein, which has an inserted amino acid sequence of seven contiguous amino acids between amino acids 591 and 592 of the wide-type VP1 of AAV1, between amino acids 588 and 589 of the wide-type VP1 of AAV9,or the corresponding position in the capsid protein of another AAV serotype than AAV1.

According to some embodiments, the rAAV vector may comprise a capsid protein which has an amino acid sequence selected from a group consisting of VNMHTRP (SEQ ID NO: 1), HATGSPR (SEQ ID NO: 2), VLTATRP (SEQ ID NO: 3), VITPTRP (SEQ ID NO: 4), VNEPRRP (SEQ ID NO: 5), VNNKTRP (SEQ ID NO: 6), WPPKTTS (SEQ ID NO: 7), PTSKTTS (SEQ ID NO: 8), LMVKTTS (SEQ ID NO: 9), WTDKTTS (SEQ ID NO: 10), QRPPREP (SEQ ID NO: 11), TAFPREP (SEQ ID NO: 12), LMTPPKTTSAQ (SEQ ID NO: 19), ATEPPKTTSAQ (SEQ ID NO: 20), AVLSPKTTSAQ (SEQ ID NO: 21), AQQRPPRPADQ (SEQ ID NO: 22), and APARPPREPAQ (SEQ ID NO: 23).

According to some embodiments, the rAAV vector provided by the present disclosure comprises a capsid protein, which has an inserted amino acid sequence selected from a group consisting of VNMHTRP (SEQ ID NO: 1), VLTATRP (SEQ ID NO: 3), VITPTRP (SEQ ID NO: 4), VNEPRRP (SEQ ID NO: 5) and VNNKTRP (SEQ ID NO: 6), between amino acids 591 and 592 of the wide-type VP1 of AAV1, or the corresponding position in the capsid protein of another AAV serotype than AAV1.

According to some embodiments, the rAAV vector provided by the present disclosure comprises a capsid protein, which has an inserted amino acid sequence selected from a group consisting of WPPKTTS (SEQ ID NO: 7), PTSKTTS (SEQ ID NO: 8), LMVKTTS (SEQ ID NO: 9), WTDKTTS (SEQ ID NO: 10), QRPPREP (SEQ ID NO: 11) and TAFPREP (SEQ ID NO: 12), between amino acids 588 and 589 of the wide-type VP1 of AAV9, or the corresponding position in the capsid protein of another AAV serotype than AAV9.

According to further embodiments, the rAAV vector provided by the present disclosure comprises a capsid protein which has an inserted amino acid sequence selected from a group consisting of AQWPPKTTSAQ (SEQ ID NO: 13), AQPTSKTTSAQ (SEQ ID NO: 14), AQLMVKTTSAQ (SEQ ID NO: 15), AQWTDKTTSAQ (SEQ ID NO: 16), AQQRPPREPAQ (SEQ ID NO: 17), AQTAFPREPAQ (SEQ ID NO: 18), LMTPPKTTSAQ (SEQ ID NO: 19), ATEPPKTTSAQ (SEQ ID NO: 20), AVLSPKTTSAQ (SEQ ID NO: 21), AQQRPPRPADQ (SEQ ID NO: 22) and APARPPREPAQ (SEQ ID NO: 23), between amino acids 586 to 591 of the wide-type VP1 of AAV9, or the corresponding position in the capsid protein of another AAV serotype than AAV9.

In some embodiments, the AAV serotypes may comprise AAV1, AAV2, AAV3A, AAV3B, AAV4, AAV5, AAV6, AAV7, AAV8, AAV9, AAV10 and the like. According to some embodiments, the rAAV vector provided by the present disclosure may be derived from AAV type 1, AAV type 2, AAV type 3A, AAV type 3B, AAV type 4, AAV type 5, AAV type 6, AAV type 7, AAV type 8, AAV type 9 or AAV type 10. According to specific embodiments, the rAAV vector provided by the present disclosure may be derived from AAV type 9.

In some embodiments, the inserted amino acid sequence may be located between amino acids 591 and 592 of the wide-type VP1 of AAV1. In some embodiments, the inserted amino acid sequence may be located between amino acids 587 and 588 of the wide-type VP1 of AAV2. In some embodiments, the inserted amino acid sequence may be located between amino acids 588 and 589 of the wide-type VP1 of AAV3A. In some embodiments, the inserted amino acid sequence may be located between amino acids 588 and 589 of the wide-type VP1 of AAV3B. In some embodiments, the inserted amino acid sequence may be located between amino acids 584 and 585 of the wide-type VP1 of AAV4. In some embodiments, the inserted amino acid sequence may be located between amino acids 575 and 576 of the wide-type VP1 of AAV5. In some embodiments, the inserted amino acid sequence may be located between amino acids 591 and 592 of the wide-type VP1 of AAV6. In some embodiments, the inserted amino acid sequence may be located between amino acids 589 and 590 of the wide-type VP1 of AAV7. In some embodiments, the inserted amino acid sequence may be located between amino acids 591 and 592 of the wide-type VP1 of AAV8. In some embodiments, the inserted amino acid sequence may be located between amino acids 588 and 589 of the wide-type VP1 of AAV9. In some embodiments, the inserted amino acid sequence may be located between amino acids 588 and 589 of the wide-type VP1 of AAV10.

In some embodiments, the wide-type VP1 of AAV1 has an amino acid sequence as shown by SEQ ID NO: 24. In some embodiments, the wide-type VP1 of AAV9 has an amino acid sequence as shown by SEQ ID NO: 31.

In some embodiments, the rAAV comprises VP1 capsid protein having an amino acid sequence as shown by SEQ ID NO: 25 or an amino acid sequence having at least 85%, 90%, 95%, 98% or 99% sequence identity thereof. In some embodiments, the rAAV comprises VP1 capsid protein having an amino acid sequence as shown by SEQ ID NO: 26 or an amino acid sequence having at least 85%, 90%, 95%, 98% or 99% sequence identity thereof. In some embodiments, the rAAV comprises VP1 capsid protein having an amino acid sequence as shown by SEQ ID NO: 27 or an amino acid sequence having at least 85%, 90%, 95%, 98% or 99% sequence identity thereof. In some embodiments, the rAAV comprises VP1 capsid protein having an amino acid sequence as shown by SEQ ID NO: 28 or an amino acid sequence having at least 85%, 90%, 95%, 98% or 99% sequence identity thereof. In some embodiments, the rAAV comprises VP1 capsid protein having an amino acid sequence as shown by SEQ ID NO: 29 or an amino acid sequence having at least 85%, 90%, 95%, 98% or 99% sequence identity thereof. In some embodiments, the rAAV comprises VP1 capsid protein having an amino acid sequence as shown by SEQ ID NO: 30 or an amino acid sequence having at least 85%, 90%, 95%, 98% or 99% sequence identity thereof. In some embodiments, the rAAV comprises VP1 capsid protein having an amino acid sequence as shown by SEQ ID NO: 32 or an amino acid sequence having at least 85%, 90%, 95%, 98% or 99% sequence identity thereof. In some embodiments, the rAAV comprises VP1 capsid protein having an amino acid sequence as shown by SEQ ID NO: 33 or an amino acid sequence having at least 85%, 90%, 95%, 98% or 99% sequence identity thereof. In some embodiments, the rAAV comprises VP1 capsid protein having an amino acid sequence as shown by SEQ ID NO: 34 or an amino acid sequence having at least 85%, 90%, 95%, 98% or 99% sequence identity thereof. In some embodiments, the rAAV comprises VP1 capsid protein having an amino acid sequence as shown by SEQ ID NO: 35 or an amino acid sequence having at least 85%, 90%, 95%, 98% or 99% sequence identity thereof. In some embodiments, the rAAV comprises VP1 capsid protein having an amino acid sequence as shown by SEQ ID NO: 36 or an amino acid sequence having at least 85%, 90%, 95%, 98% or 99% sequence identity thereof. In some embodiments, the rAAV comprises VP1 capsid protein having an amino acid sequence as shown by SEQ ID NO: 37 or an amino acid sequence having at least 85%, 90%, 95%, 98% or 99% sequence identity thereof. In some embodiments, the rAAV comprises VP1 capsid protein having an amino acid sequence as shown by SEQ ID NO: 38 or an amino acid sequence having at least 85%, 90%, 95%, 98% or 99% sequence identity thereof. In some embodiments, the rAAV comprises VP1 capsid protein having an amino acid sequence as shown by SEQ ID NO: 39 or an amino acid sequence having at least 85%, 90%, 95%, 98% or 99% sequence identity thereof. In some embodiments, the rAAV comprises VP1 capsid protein having an amino acid sequence as shown by SEQ ID NO: 40 or an amino acid sequence having at least 85%, 90%, 95%, 98% or 99% sequence identity thereof. In some embodiments, the rAAV comprises VP1 capsid protein having an amino acid sequence as shown by SEQ ID NO:41 or an amino acid sequence having at least 85%, 90%, 95%, 98% or 99% sequence identity thereof. In some embodiments, the rAAV comprises VP1 capsid protein having an amino acid sequence as shown by SEQ ID NO: 42 or an amino acid sequence having at least 85%, 90%, 95%, 98% or 99% sequence identity thereof.

According to some embodiments, the CAR comprises an antigen-binding domain which specifically binds to a CNS tumor cell. According to some embodiments, the CAR may specifically bind to a solid CNS tumor, for example, but not limit to, gliomas, glioneuronal tumors, neuronal tumors, such as adult-type diffuse gliomas (e.g., astrocytoma, oligodendroglioma, glioblastoma), pediatric-type diffuse low-grade gliomas (e.g. diffuse astrocytoma, angiocentric glioma, polymorphous low-grade neuroepithelial tumor of the young, diffuse low-grade glioma), pediatric-type diffuse high-grade gliomas (e.g. diffuse midline glioma, diffuse hemispheric glioma, diffuse pediatric-type high-grade glioma, infant-type hemispheric glioma), circumscribed astrocytic gliomas (e.g. pilocytic astrocytoma, high-grade astrocytoma with piloid features, pleomorphic xanthoastrocytoma, subependymal giant cell astrocytoma, chordoid glioma, astroblastoma), glioneuronal and neuronal tumors (e.g. ganglioglioma, desmoplastic infantile ganglioglioma/desmoplastic infantile astrocytoma, dysembryoplastic neuroepithelial tumor, diffuse glioneuronal tumor with oligodendroglioma-like features and nuclear clusters, papillary glioneuronal tumor, rosette-forming glioneuronal tumor, myxoid glioneuronal tumor, diffuse leptomeningeal glioneuronal tumor, gangliocytoma, multinodular and vacuolating neuronal tumor, dysplastic cerebellar gangliocytoma (Lhermitte-Duclos disease), central neurocytoma, extraventricular neurocytoma, cerebellar liponeurocytoma), ependymal tumors (e.g. supratentorialependymoma, supratentorial ependymoma, supratentorial ependymoma, posterior fossa ependymoma, posterior fossa ependymoma, posterior fossa ependymoma, spinal ependymoma, spinal ependymoma, myxopapillary ependymoma, Subependymoma); choroid plexus tumors, such as choroid plexus papilloma, atypical choroid plexus papilloma, and choroid plexus carcinoma; embryonal tumors, such as medulloblastoma, atypical teratoid/rhabdoid tumor, cribriform neuroepithelial tumor, embryonal tumor with multilayered rosettes CNS neuroblastoma, CNS tumor with BCOR internal tandem duplication, and CNS embryonal tumor; pineal tumors, such as pineocytoma, pineal parenchymal tumor of intermediate differentiation, pineoblastoma, papillary tumor of the pineal region, and desmoplastic myxoid tumor of the pineal region; cranial and paraspinal nerve tumors, such as schwannoma, neurofibroma, perineurioma, hybrid nerve sheath tumor, malignant melanotic nerve sheath tumor, malignant peripheral nerve sheath tumor, and paraganglioma; meningiomas; mesenchymal non-meningothelial tumors, such as soft tissue tumors (e.g. fibroblastic and myofibroblastic tumors such as solitary fibrous tumor, vascular tumors such as hemangiomas and vascular malformations and hemangioblastoma, skeletal muscle tumors such as rhabdomyosarcoma, uncertain differentiation such as intracranial mesenchymal tumor, CIC-rearranged sarcoma, primary intracranial sarcoma, ewing sarcoma), and chondro-osseous tumors (e.g., chondrogenic tumors such as mesenchymal chondrosarcoma chondrosarcoma, notochordal tumors such as chordoma (including poorly differentiated chordoma)); melanocytic tumors, such as diffuse meningeal melanocytic neoplasms (e.g. meningeal melanocytosis and meningeal melanomatosis) and circumscribed meningeal melanocytic neoplasms (e.g. meningeal melanocytoma and meningeal melanoma); germ cell tumors, such as mature teratoma, immature teratoma, teratoma with somatic-type malignancy, germinoma, embryonal carcinoma, yolk sac tumor, choriocarcinoma, and mixed germ cell tumor; tumors of the sellar region, such as adamantinomatous craniopharyngioma, papillary craniopharyngioma, pituicytoma, granular cell tumor of the sellar region, and spindle cell oncocytoma, pituitary adenoma/PitNET, and pituitary blastoma; and metastases to the CNS, such as metastases to the brain and spinal cord parenchyma, and metastases to the meninges.

According to some embodiments, the CAR may specifically bind to tumor-associated antigens (TAAs) of the solid CNS tumor, for example, but not limit to, B7-H1, B7-H3 (also known as CD276), B7-H4, B7-H5, B7-H7, BT3.1 (also known as BTF5 or CD277); natural-killer 2 receptor (NKR2); natural-killer group 2, member D receptor protein (NKG2D); CD19; CD48; CD133; carcinoembryonic antigen (CEA); epidermal growth factor receptor (EGFR); epidermal growth factor receptor variant III (EGFRvIII); epithelial cellular adhesion molecule (EpCAM); mucin 1 (MUC1); epidermal growth factor receptor 2 (HER2); interleukin 13 receptor α2 (IL13Rα2); EPH Receptor A2 (GD3, A2); and Disialoganglioside 2 (GD2), GD3, mesothelin, Tn Ag, PSMA, TAG72, CD44v6, KIT, leguman, CD171, IL-1 IRa, PSCA, MAD-CT-1, MAD-CT-2, VEGFR2, LewisY, CD24, PDGFR-beta, SSEA-4, folate receptor alpha, ERBBs (e g., ERBB2), NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, Polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, GPR20, Ly6k, OR51E2, TARP, or GFRa4. In some preferred embodiments, the TAAs of the solid CNS tumor may be B7-H3.

According to some embodiments, the CAR may comprise, from N-terminus to C-terminus, an antigen-binding domain, a hinge domain, a transmembrane domain (TMD) and an intracellular signaling domain (ICD).

According to some embodiments, the TMD may be derived from a polypeptide selected from a T-cell receptor (TCR) alpha chain, a TCR beta chain, a TCR zeta chain, CD3 epsilon, CD4, CD5,CD8, CD9, CD16, CD22, CD27 (TNFRSF19), CD28, CD33, CD45, CD80, CD83, CD86, CD134, CD137, CD152 (CTLA4), CD154, CD279, PD-1, and a combination of any thereof. According to some embodiments, the ICD may comprise a co-stimulatory domain. According to a specific embodiment, the TMD comprise an amino acid sequence as shown by SEQ ID NO:44 or an amino acid sequence having at least 85%, 90%, 95%, 98% or 99% sequence identity thereof.

According to some embodiments, the ICD may comprise a first intracellular signaling domain derived from the group consisting of 4-1BB (CD137), CD27 (TNFRSF7), CD28, OX40 (CD 134), CD70, LFA-2 (CD2), CD5, ICAM-1 (CD54), LFA-1 (CD1 la/CD18), DAPIO, DAP12, a co-stimulatory inducible T-cell costimulatory (ICOS) polypeptide sequence, and a combination of any thereof. According to a specific embodiment, the first intracellular signaling domain comprise an amino acid sequence as shown by SEQ ID NO:45 or an amino acid sequence having at least 85%, 90%, 95%, 98% or 99% sequence identity thereof.

According to some embodiments, the ICD may further comprise a second intracellular signaling domain derived from of CD3 zeta, of FCGR3A and of NKG2D, and a combination of any thereof. According to a specific embodiment, the second intracellular signaling domain comprise an amino acid sequence as shown by SEQ ID NO: 46 or an amino acid sequence having at least 85%, 90%, 95%, 98% or 99% sequence identity thereof.

According to some embodiments, the hinge domain may comprise a polypeptide derived from CD8. According to some embodiments, the hinge domain may comprise an amino acid sequence as shown by SEQ ID NO: 43 or an amino acid sequence having at least 95%, 98% or 99% sequence identity thereof.

According to some embodiments, the CAR may comprise an antigen-binding domain which specifically binds to the tumor-associated antigens (TAAs) of the solid CNS tumor. According to some embodiments, the antigen-binding domain of the CAR may comprises an antibody, an antibody fragment, an scFv, a Fv, a Fab, a (Fab′)2, a single domain antibody (SDAB), a VH or VL domain, a camelid VHH domain or a bi-functional (e.g. bispecific) hybrid antibody.

According to another aspect, provided is a modified cell, particular a modified microglia and/or astrocyte, which expresses a chimeric antigen receptor (CAR) specifically binding to a tumor cell.

According to some embodiments, the modified cell may be a microglial cell. According to some embodiments, the modified cell may be astrocyte. According to some embodiments, the modified cell may be iPSC-derived microglia like cell. According to some embodiments, the modified cell may be monocyte-derived microglia-like cell.

According to some embodiments, the CAR may be introduced into the cell by means of the rAAV vector of the present disclosure.

According to some embodiments, the expression level of the CAR in the modified cell can be further increased by pharmacological approaches. In a certain embodiments, the topoisomerase and proteasome inhibitor is used for further increasing the expression level of the heterologous nucleotide sequence, which is transduced by using the rAAV vector of the present disclosure. According to a preferred embodiment, a topoisomerase inhibitor, e.g. doxorubicin, may be used for increasing the expression level of the CAR.

According to some embodiments, the modified cell, particularly the modified microglial cell, may be not activated by the infection of the rAAV of the present disclosure.

According to some embodiments, the modified cell, particularly the modified microglial cell, may recognize tumor cells, e.g. CNS tumor cells.

According to some embodiments, the modified microglial cell may be activated as contacting the tumor cells, e.g. CNS tumor cells. According to some embodiments, the modified microglial cell may be capable of phagocytosing the tumor cells.

According to yet another aspect, provided is a method for obtaining the above modified cells. According to some embodiments, the method comprises a step of transducing microglia and/or astrocytes with the rAAV of the present disclosure.

According to yet another aspect, provided is a pharmaceutical composition comprising the rAAV vector of the present disclosure, or the modified cell of the present disclosure. According to some embodiments, the pharmaceutical composition may further comprise a pharmaceutically acceptable excipient.

According to yet another aspect, provided is use of the rAAV vector, the modified cell or the pharmaceutical composition of the present disclosure for treating a tumor, particular a CNS tumor.

According to yet another aspect, provided is a method for treating a CNS tumor, particular a solid CNS tumor, which comprises administering a therapeutically effective amount of the rAAV vector, the modified cell or the pharmaceutical composition of the present disclosure to a subject in need thereof.

According to some embodiments, the pharmaceutical composition of the present disclosure may be used for treating a solid CNS tumor, for example, but not limit to, gliomas, glioneuronal tumors, neuronal tumors, such as adult-type diffuse gliomas (e.g., astrocytoma, oligodendroglioma, glioblastoma), pediatric-type diffuse low-grade gliomas (e.g. diffuse astrocytoma, angiocentric glioma, polymorphous low-grade neuroepithelial tumor of the young, diffuse low-grade glioma), pediatric-type diffuse high-grade gliomas (e.g. diffuse midline glioma, diffuse hemispheric glioma, diffuse pediatric-type high-grade glioma, infant-type hemispheric glioma), circumscribed astrocytic gliomas (e.g. pilocytic astrocytoma, high-grade astrocytoma with piloid features, pleomorphic xanthoastrocytoma, subependymal giant cell astrocytoma, chordoid glioma, astroblastoma), glioneuronal and neuronal tumors (e.g. ganglioglioma, desmoplastic infantile ganglioglioma/desmoplastic infantile astrocytoma, dysembryoplastic neuroepithelial tumor, diffuse glioneuronal tumor with oligodendroglioma-like features and nuclear clusters, papillary glioneuronal tumor, rosette-forming glioneuronal tumor, myxoid glioneuronal tumor, diffuse leptomeningeal glioneuronal tumor, gangliocytoma, multinodular and vacuolating neuronal tumor, dysplastic cerebellar gangliocytoma (Lhermitte-Duclos disease), central neurocytoma, extraventricular neurocytoma, cerebellar liponeurocytoma), ependymal tumors (e.g. supratentorialependymoma, supratentorial ependymoma, supratentorial ependymoma, posterior fossa ependymoma, posterior fossa ependymoma, posterior fossa ependymoma, spinal ependymoma, spinal ependymoma, myxopapillary ependymoma, Subependymoma); choroid plexus tumors, such as choroid plexus papilloma, atypical choroid plexus papilloma, and choroid plexus carcinoma; embryonal tumors, such as medulloblastoma, atypical teratoid/rhabdoid tumor, cribriform neuroepithelial tumor, embryonal tumor with multilayered rosettes CNS neuroblastoma, CNS tumor with BCOR internal tandem duplication, and CNS embryonal tumor; pineal tumors, such as pineocytoma, pineal parenchymal tumor of intermediate differentiation, pineoblastoma, papillary tumor of the pineal region, and desmoplastic myxoid tumor of the pineal region; cranial and paraspinal nerve tumors, such as schwannoma, neurofibroma, perineurioma, hybrid nerve sheath tumor, malignant melanotic nerve sheath tumor, malignant peripheral nerve sheath tumor, and paraganglioma; meningiomas; mesenchymal non-meningothelial tumors, such as soft tissue tumors (e.g. fibroblastic and myofibroblastic tumors such as solitary fibrous tumor, vascular tumors such as hemangiomas and vascular malformations and hemangioblastoma, skeletal muscle tumors such as rhabdomyosarcoma, uncertain differentiation such as intracranial mesenchymal tumor, CIC-rearranged sarcoma, primary intracranial sarcoma, ewing sarcoma), and chondro-osseous tumors (e.g., chondrogenic tumors such as mesenchymal chondrosarcoma chondrosarcoma, notochordal tumors such as chordoma (including poorly differentiated chordoma)); melanocytic tumors, such as diffuse meningeal melanocytic neoplasms (e.g. meningeal melanocytosis and meningeal melanomatosis) and circumscribed meningeal melanocytic neoplasms (e.g. meningeal melanocytoma and meningeal melanoma); germ cell tumors, such as mature teratoma, immature teratoma, teratoma with somatic-type malignancy, germinoma, embryonal carcinoma, yolk sac tumor, choriocarcinoma, and mixed germ cell tumor; tumors of the sellar region, such as adamantinomatous craniopharyngioma, papillary craniopharyngioma, pituicytoma, granular cell tumor of the sellar region, and spindle cell oncocytoma, pituitary adenoma/PitNET, and pituitary blastoma; and metastases to the CNS, such as metastases to the brain and spinal cord parenchyma, and metastases to the meninges.

According to some embodiments, the pharmaceutical composition of the present disclosure may be administered in a therapeutically effective amount to a subject in need thereof. According to some embodiments, the pharmaceutical composition of the present disclosure may be administered by intratumoral or paratumoral injection.

Unless defined otherwise, all technical and scientific terms used herein have the same meaning as commonly understood by those skilled in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It is noted that as used herein and in the appended claims, the singular forms “a,” “an,” and “the” include plural referents unless the context clearly dictates otherwise. Thus, for example, reference to “a recombinant AAV virion” includes a plurality of such virions and reference to “microglia” includes reference to one or more microglia cells and equivalents thereof known to those skilled in the art, and so forth.

### Definitions

Unless otherwise defined, all scientific and technical terms used herein have the same meaning as commonly understood by those skilled in the art to which this technology belongs.

Adeno-associated virus (AAV) is a member of the Parvoviridae, belonging to the Dependovirus genus. AAV is a nonpathogenic parvovirus composed of a single-stranded DNA genome of approximately 4.7 kb within a non-enveloped, icosahedral capsid. The genome contains three open reading frames (ORF) flanked by inverted terminal repeats (ITR) that function as the viral origin of replication and packaging signal. The rep ORF encodes four nonstructural proteins that play roles in viral replication, transcriptional regulation, site-specific integration, and virion assembly. The cap ORF encodes three structural proteins (VPs 1-3) that assemble to form a 60-mer viral capsid. Finally, an ORF present as an alternate reading frame within the cap gene produces the assembly-activating protein (AAP), a viral protein that localizes AAV capsid proteins to the nucleolus and functions in the capsid assembly process. Based on crystal structures of AAV, the VP amino acids involved in forming the icosahedral fivefold, threefold, and twofold symmetry interfaces have been visualized. The surface loops at the threefold axis of symmetry are thought to be involved in host cell receptor binding and have been the target of mutagenesis studies.

There are several naturally occurring (“wild-type”) serotypes and over 100 known variants of AAV, each of which differs in amino acid sequence, particularly within the hypervariable regions of the capsid proteins, and thus in their gene delivery properties. No AAV has been associated with any human disease, making recombinant AAV attractive for clinical applications.

Three AAV capsid proteins (i.e., VP1, VP2 and VP3) are produced in an overlapping fashion from the cap ORF by using alternative mRNA splicing of the transcript and alternative translational start codon usage. A common stop codon is employed for all three capsid proteins. Though only VP1 is illustrated in the examples and drawings, it should be understood that each of VP1, VP2 and VP3 comprises the inserted amino acid sequence of seven contiguous amino acids of the present disclosure.

Otherwise indicated, the term “adeno-associated virus” or “AAV” refers to all subtypes or serotypes and both replication-competent and recombinant forms. The term “AAV” includes, without limitation, AAV type 1 (AAV-1 or AAV1), AAV type 2 (AAV-2 or AAV2), AAV type 3A (AAV-3A or AAV3A), AAV type 3B (AAV-3B or AAV3B), AAV type 4 (AAV-4 or AAV4), AAV type 5 (AAV-5 or AAV5), AAV type 6 (AAV-6 or AAV6), AAV type 7 (AAV-7 or AAV7), AAV type 8 (AAV-8 or AAV8), AAV type 9 (AAV-9 or AAV9), AAV type 10 (AAV-10 or AAV 10 or AAVrh10), avian AAV, bovine AAV, canine AAV, caprine AAV, equine AAV, primate AAV, non-primate AAV, and ovine AAV. “Primate AAV” refers to AAV that infect primates, “non-primate AAV” refers to AAV that infect non-primate mammals, “bovine AAV” refers to AAV that infect bovine mammals and the like.

The genomic sequences of various serotypes of AAV, as well as the sequences of the native terminal repeats (TRs), Rep proteins, and capsid subunits are known in the art. Such sequences may be found in the literature or in public databases such as GenBank. See, e.g., GenBank Accession Numbers NC_002077.1 (AAV1), AF063497.1 (AAV1), NC_001401.2 (AAV2), AF043303.1 (AAV2), J01901.1 (AAV2), U48704.1 (AAV3A), NC_001729.1 (AAV3A), AF028705.1 (AAV3B), NC.001829.1 (AAV4), U89790.1 (AAV4), NC_006152.1 (AA5), AF085716.1 (AAV-5), AF028704.1 (AAV6), NC 006260.1 (AAV7), AF513851.1 (AAV7), AF513852.1 (AAV8) NC 006261.1 (AAV-8), AY530579.1 (AAV9), AAT46337 (AAV10) and AAO88208 (AAVrh10); the disclosures of which are incorporated by reference herein for teaching AAV polynucleotide and amino acid sequences.

The term “recombinant adeno-associated virus capsid protein” or “rAAV capsid protein” as used herein refers to an AAV capsid protein comprising a seven-amino-acid peptide insertion in a GH-loop of the VP1-VP3 capsid protein as compared to a wide-type VP1-VP3 capsid protein thereof.

The AAV variants disclosed herein were generated at least in part through the use of in vitro or in vivo directed evolution methodology, such as the techniques described above, involving the use of screening in cultured primary mouse microglia cells or in vivo microglia following injecting into the striatum and/or midbrain of the mice. As such, the AAV variant capsids disclosed herein comprise a seven-amino-acid peptide insertion in a GH-loop of VP1, VP2 and/or VP3 that confer more efficient transduction than a corresponding parental AAV capsid protein or control. As used herein, a “corresponding parental AAV capsid protein” refers to an AAV capsid protein of the same wild-type or variant AAV serotype as the subject variant AAV capsid protein but that does not comprise the peptide insertion of the subject variant AAV capsid protein.

The term “recombinant adeno-associated virus virion(s)”, “rAAV virion(s)”, “rAAV vector(s)” or “rAAV particles” as used herein refers to a viral particle comprising a recombinant/variant capsid protein.

The term “sequence identity” or “identity” as used herein means the percentage of pair-wise identical residues-following (homologous) alignment of a sequence of a polypeptide of the disclosure with respect to the number of residues in the longer of these two sequences. Sequence identity is measured by dividing the number of identical amino acid residues by the total number of residues and multiplying the product by 100.

If an AAV vector/virion comprises a heterologous polynucleotide sequence, the heterologous polynucleotide sequence refers to a polypolynucleotide sequence other than a wild-type AAV genome, e.g., a transgene to be delivered to a target cell. In general, the heterologous polynucleotide sequence is flanked by at least one, and generally by two, AAV inverted terminal repeat sequences (ITRs).

The term “packaging” or “package” as used herein refers to a series of intracellular events that result in the assembly and encapsidation of an AAV particle. AAV “rep” and “cap” genes refer to polypolynucleotide sequences encoding replication and encapsidation proteins of adeno-associated virus. AAV rep and cap are referred to herein as AAV “packaging genes”.

The term “polynucleotide” as used herein refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double-and single-stranded molecules. Unless otherwise specified or required, any embodiment herein that comprises a polynucleotide encompasses both the double-stranded form and each of two complementary single-stranded forms known or predicted to make up the double-stranded form.

The terms “treatment”, “treating” and the like as used herein, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease.

The terms “individual”, “host”, “subject”, and “patient” are used interchangeably herein, and refer to a mammal, including, but not limited to, humans; non-human primates, including simians; mammalian sport animals (e.g., horses); mammalian farm animals (e.g., sheep, goats, etc.); mammalian pets (dogs, cats, etc.); and rodents (e.g., mice, rats, etc.).

The term “Chimeric Antigen Receptor” or alternatively a “CAR” refers to a recombinant polypeptide construct comprising at least an extracellular antigen binding domain, a transmembrane domain and a cytoplasmic signaling domain (also referred to herein as “an intracellular signaling domain”) comprising a functional signaling domain derived from a stimulatory molecule as defined below. In some embodiments, the domains in the CAR polypeptide construct are in the same polypeptide chain, e.g., comprise a chimeric fusion protein. In some embodiments, the domains in the CAR polypeptide construct are not contiguous with each other, e.g., are in different polypeptide chains, e.g., as provided in an RCAR as described herein.

The term “microglia” or “microglial cell(s)” as used herein means the cells of mesodermal/mesenchymal origin that migrate into the CNS to become resident macrophages within the unique brain microenvironment. Microglia are highly dynamic cells that interact with neurons and non-neuronal cells. Microglia patrol the brain parenchyma via continuous process extension and retraction and are also capable of transitioning from a ramified to an ameboid morphology, a feature that is consistent with cell activation. Microglia express a wide array of receptors and thus respond to pleiotropic stimuli ranging from neurotransmitters to cytokines and plasma proteins. They play a crucial role in the healthy brain as regulators of synaptic functions and phagocytosis of newborn neurons, with important implications in synaptic plasticity and adult neurogenesis. In disease, they play a crucial role in neurological and neuroinflammatory conditions. Their interactions with T cells are a major component of the development of brain autoimmunity, while their pathogenic interactions with neurons via induction of ROS and iNOS play a crucial role in neurological disorders. Emerging genetic tools and animal models have shed new light on the origin of microglia, their link to peripheral monocytes, and their contribution to disease pathogenesis. As microglia might exert beneficial and pathogenic functions in the CNS, understanding their contribution in disease-specific contexts will be necessary for the identification of novel microglia-targeted therapies for CNS diseases.

The term “induced pluripotent stem cells” or “iPSCs” as used herein is meant a cell derived from skin or blood cells that has been reprogrammed back into an embryonic-like pluripotent state to enable the development of an unlimited source of any type of cell needed for therapeutic purposes. In some embodiments, iPSCs can be differentiated into microglial cells by any well-known approaches.

The term “monocytes” as used herein means a type of white blood cell, or leukocyte. Monocytes are the largest type of leukocyte and can differentiate into macrophages and myeloid lineage dendritic cells. As a part of the vertebrate innate immune system monocytes also influence the process of adaptive immunity. Monocytes compose 2% to 10% of all leukocytes in the human body and serve multiple roles in immune function. Such roles include, without limitation: replenishing resident macrophages under normal conditions; migration within approximately 8-12 hours in response to inflammation signals from sites of infection in the tissues; and differentiation into macrophages or dendritic cells to affect an immune response.

The term “directed evolution” as used herein refers to a capsid engineering methodology, in vitro and/or in vivo, which emulates natural evolution through iterative rounds of genetic diversification and selection processes, thereby accumulating beneficial mutations that progressively improve the function of a biomolecule. Directed evolution often involves an in vivo method referred to as “biopanning” for selection of AAV variants from a library which variants possess a more efficient level of infectivity of a cell or tissue type of interest.

With regards to cell modification, the term “genetically modified” or “transformed” or “transfected” or “transduced” by exogenous DNA (e.g. via a recombinant virus) refers to when such DNA has been introduced inside the cell. The presence of the exogenous DNA results in permanent or transient genetic change. The transforming DNA may or may not be integrated (covalently linked) into the genome of the cell.

The following examples are provided for assisting the understanding of the present disclosure. It should be understood that these examples are only used to illustrate the present invention, but do not constitute any limitation. Any modifications and changes may be made without departing from the spirit of the invention.

### Materials and Methods

Mice. Animal care and use followed the approval of the Animal Care and Use Committee of the National Institute of Biological Sciences, Beijing in accordance with the Regulations for the Administration of Affairs Concerning Experimental Animals of China. Cx3cr1^{CreER} mice (021160, Cx3cr1^{tm2.1(cre/ERT2)Litt}/WganJ), Rosa26-LSL-DTA mice (009669, Gt (ROSA)26Sortm1(DTA)Lky/J) and Cx3cr1^{GFP} mice (005582, Cx3cr1^{tm1Litt}/J) were obtained from Jackson Laboratory. Cx3cr1^{CreER} mice and Rosa26-LSL-DTA mice were bred to obtain Cx3cr1^{CreER}: Rosa26-LSL-DTA mice that were used for microglia replacement and CAR-Mi therapy for glioblastoma study. Adult mice of either sex were used for in vivo studies. The postnatal day 1 (P1) and adult C57BL/6N wildtype mice were obtained from Beijing Vital River Laboratory Animal Technology. Mice were maintained with a 12/12 hour photoperiod (light on at 8 AM) and were provided food and water ad libitum.

Plasmids. The plasmids for capsid screening were constructed according to the CREATE protocol with modifications. The pAAV-CMV-mScarlet-ΔCap1-DIO-SV40pA plasmid contains an mScarlet expression cassette, an in cis Cap cassette, and a DIO cassette. The mScarlet expression cassette consists of a CMV promoter, the mScarlet coding sequence, and a SV40 pA sequence. The in cis Cap cassette includes the AAV5 p41 promoter sequence, the AAV2 rep splicing sequence, and the AAV1 cap sequence. The AAV1 cap sequence was modified for subsequent library generation. The DIO cassette contains a SV40pA sequence. The pCap1-T plasmid contains the DNA sequences of AA448-591 of AAV1 cap. The pCap1-T-mut plasmid contains the DNA sequences of AA448-589 of AAV1 cap. The AAV2/9 REP-AAP helper plasmid was constructed following the original report.

Similarly, the pAAV-CMV-mScarlet-ΔCap9-DIO-SV40pA plasmid is constructed. The in cis Cap cassette includes the AAV5 p41 promoter sequence, the AAV2 rep splicing sequence, and the AAV9 cap sequence. The AAV9 cap sequence was modified to introduce XbaI and AgeI sites for subsequent library generation. The pCRII-9Cap-xE plasmid was constructed following the original report.

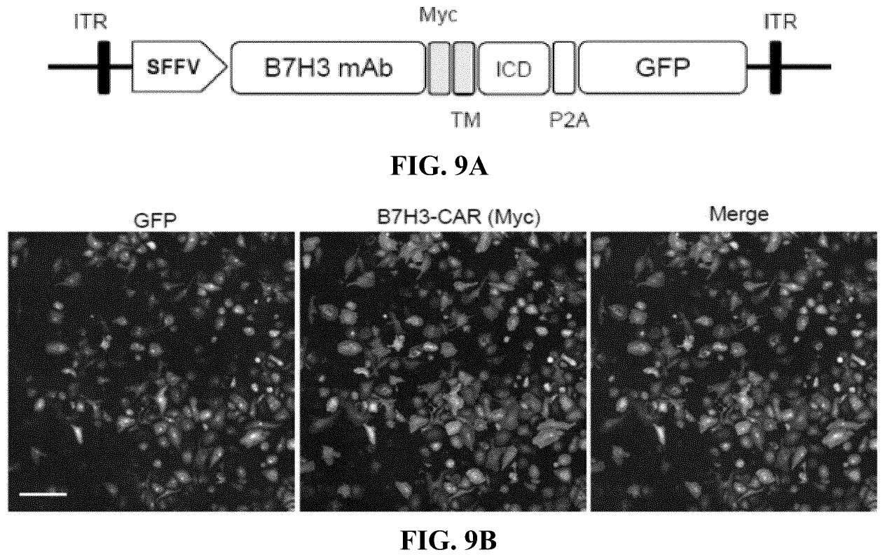

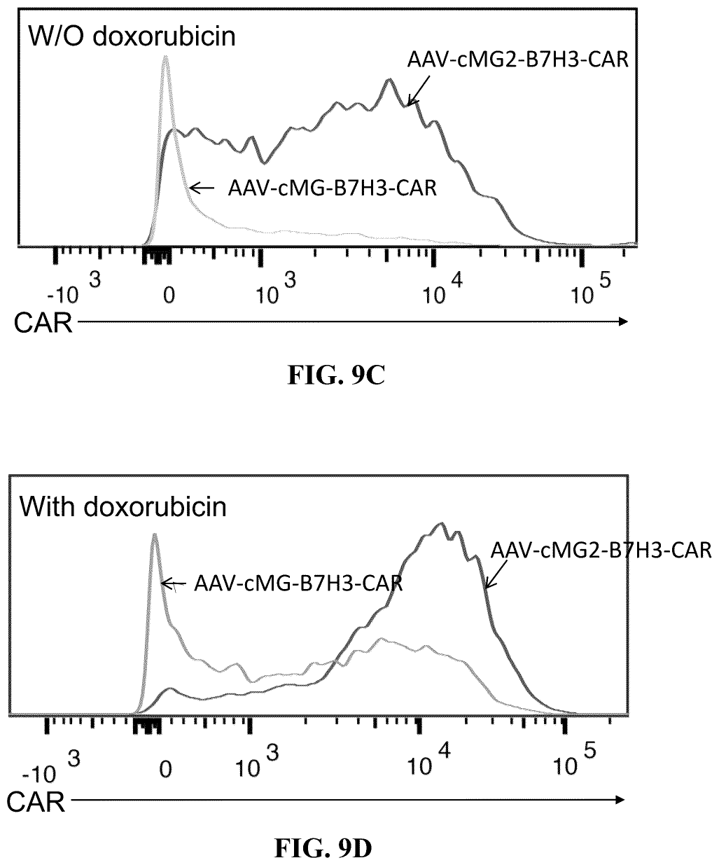

The B7H3-CAR comprises of (from N-to C-terminal) a B7H3 targeting monoclonal antibody (mAb, 2E6), a CD8 hinge region, a Myc tag, a CD8 transmembrane domain, a 4-1BB costimulatory domain, and a CD3 zeta cytoplasmic domain. The B7H3-CAR coding sequence was synthesized and cloned into the pAAV-SFFV backbone together with the coding sequence for P2A and GFP (pAAV-SFFV-B7H3-CAR-2A-GFP; FIG. 9A). The B7H3-CAR-ΔICDs was generated by replacing the 4-1BB costimulatory domain and the CD3 zeta cytoplasmic domain with a HA tag. The CD19-CAR was generated by replacing the B7H3 targeting mAb with a CD19-targeting mAb. To display human B7H3 ECDs, the coding sequence for the ECD and the transmembrane domain of human B7H3 (amino acid 1-274) was amplified by PCR from the cDNA of U87 cells, and was cloned into the pLJM1-EGFP vector together with the coding sequence for P2A and mCherry or together with the coding sequence for P2A and Firefly luciferase.

AAV packaging. AAV vectors were packaged as previously described. Briefly, the AAV vectors and the AAV helper plasmids were co-transfected into HEK293T cells. Cells were harvested 96 hours after transfection, and the viral particles were released from cells by freeze-thaw cycles and sonication. The virus was purified using cesium chloride density-gradient ultracentrifugation and dialyzed into phosphate-buffered saline (PBS) buffer. The viral titer was determined by qPCR.

Cell lines. The U87 and GL261 cell lines were obtained from the American Type Culture Collection (ATCC). GL261 cell lines were transduced with a lentiviral vector co-encoding the human B7H3 ECD and mCherry or a lentiviral vector co-encoding the human B7H3 ECD and Firefly luciferase. U87 cell lines were transduced with a lentiviral vector encoding mCherry. U87 and GL261 cell lines were grown in DMEM supplemented with 10% fetal bovine serum (FBS) and 1% penicillin-streptomycin (P/S) at 37° C. in a humidified 5% CO2 incubator.

Mouse microglia isolation and culture. Primary mouse microglia cells were obtained from P1 C57BL/6 wild-type mice. Pups were placed on ice for 1-2 mins until unresponsive, then were soaked with 75% alcohol, and were carefully decapitated. Brains were collected with clean sterile scissor and placed in a 10-cm dish containing 10 mL iced dissociation medium (DMEM/F12 (11330032, Gibco) supplemented with 100 U/mL penicillin and 100 μg/mL streptomycin (P/S, 15140-122, Gibco). All meninges were removed using No.5 Dumont forceps under dissecting microscope. Brains were mechanically dissociated in dissociation medium. Dissociated cells were filtered through a 40-μm cell strainer and centrifuged at 1000 rpm for 10 mins at room temperature. Pellets were resuspended with culture medium (DMEM/F12 supplemented with 10% fetal bovine serum (FBS, 0099-141, Gibco), 5 ng/mL granulocyte-macrophage colony-stimulating factor (GM-CSF, PRP100489, Abbkine) and 1% P/S), and plated at a density of five brains per T-75 plastic culture flask (Falcon) pre-coated with poly-L-lysine (P8920, Sigma-Aldrich). The culture medium was changed 24 hours after isolation. After that, 50% culture medium was changed every 3 days. Two weeks later, the flasks were shaken at 180 rpm using an orbital shaker for 2 hours at 37° C. to harvest microglia. Cultured microglia were maintained at 37° C. in a humidified incubator with 5% CO₂.

In vitro AAV transduction. Microglia were plated in 6-well cell culture plate (6005550, PerkinElmer). Microglia were transduced with rAAVs packaged using candidate capsids at multiplicity of infection (MOI) of 10,000. After 2 days, the culture medium was changed into the TIC medium⁴ (DMEM/F12 supplemented with 1% P/S, 2 mM L-glutamine (25030-081, Gibco), 5 mg/mL N-acetyl cysteine (A9165, Sigma-Aldrich), 5 mg/mL insulin (I0516, Sigma-Aldrich), 100 mg/mL apo-transferrin (T1147, Sigma-Aldrich), 100 ng/mL sodium selenite (S5261, Sigma-Aldrich), 2 ng/mL recombinant murine TGF-ß2 (50153-M08H, Sino Biological), 100 ng/mL recombinant murine interleukin-34 (50055-M08H, Sino Biological), and 1.5 mg/mL cholesterol (ovine wool, 700000P, Merck). The subsequent assays were performed as least 5 days after rAAVs transduction.

Cultured microglia RNA sequencing. Bulk RNA sequencing of AAV-cMG2-B7H3-CAR-transduced, doxorubicin-treated AAV-cMG2-B7H3-CAR-transduced or control untransduced mouse primary microglia were performed. As additional controls, two groups of mouse primary microglia that were exposed to 200 ng/mL lipopolysaccharide (LPS, L4130, Sigma-Aldrich) or 20 ng/mL recombinant murine interleukin-4 (IL-4, 214-14, PeproTech) in TIC medium for 24 hours were also prepared for RNA sequencing. Total RNAs of treated microglia were extracted using TRIzol (15596018, Thermo Fisher Scientific) and subjected to single-end 75 bp high-throughput sequencing on an Illumina platform.

In vitro screening. The detail sequences of primers used in this study are listed in Table 1. An AAV capsid library was first constructed by inserting random heptamers into the reading frame for each capsid protein, VP 1-3, of the AAV1/AAV9 capsid using the CREATE protocol. Briefly, the library fragments were generated by PCR using the primers XF and 7xMNN with the pCRII-9Cap-xE plasmid serving as AAV9 template, and primers Cap1-insertion-F and Cap1-591-7MNN-R with the pCap1-T plasmid serving as AAV1 template. The pAAV-CMV-mScarlet-ACap1/9-DIO-SV40pA plasmid was linearized. The library fragments were assembled into the linearized pAAV-CMV-mScarlet-ΔCap1/9-DIO-SV40pA plasmid using Gibson assembly. The resulted library was packaged into rAAVs by co-transfecting the AAV capsid library, the AAV2/9 REP-AAP helper plasmid and the AAV-helper plasmid into HEK293T cells. Approximately 10 library rAAVs were used to transduce the cultured mouse microglia for 24 hours. 48 hours after transduction, the genomes of rAAVs that had successfully transduced the cultured microglia were recovered using Trizol. The cap sequences were first amplified from recovered AAV genomes by PCR using specific primers (9CapF and SV40pA-R for AAV9 library; Cap-F and SV40pA-R for AAV1 library). The PCR product was purified and used as the template for the second PCR reaction that used specific primer pairs (XF and 588i-R for AAV9 library; Cap1-insertion-F and Cap1-591i-R for AAV1 library). The recovered cap sequences were then assembled back into the pAAV-CMV-mScarlet-ΔCap1/9-DIO-SV40pA plasmid and screened again in the cultured mouse microglia. The candidates that were highly enriched after two rounds of screening were identified through next generation sequencing (NGS) and individually tested. The enrichment score of a variant was calculated as follows:

TABLE 1
Primers sequences for library construction and variant recovery.

| Name | Sequence (5′-3′) |
|---|---|
| XF | actcatcgaccaatacttgtactatctctctagaac (SEQ ID NO.: 48) |
| 7xMNN | gtattccttggttttgaacccaaccggtctgcgcctgtgcmnnmnnmnnmnnmnnmnnmnnttgggcactctggtggt |
| | ttgtg (SEQ ID NO.: 49) |
| 9CapF | caggtcttcacggactcagactatcag (SEQ ID NO.: 50) |
| CDF | cattgatgagtttggacaaaccacaactagaatg (SEQ ID NO.: 51) |
| 1527 | acactctttccctacacgacgctcttccgatctgacaagtggccacaaaccaccag (SEQ ID NO.: 52) |
| 1532 | gtgactggagttcagacgtgtgctcttccgatctccttggttttgaacccaaccg (SEQ ID NO.: 53) |
| SV40pA-R | cattctagttgtggtttgtccaaactcatcaatg (SEQ ID NO.: 54) |
| 588i-F | aatagcttgatgaatcctggacctg (SEQ ID NO.: 55) |
| 588i-R | ttccttggttttgaacccaaccg (SEQ ID NO.: 56) |
| WPP-mut-R1 | gtattccttggttttgaacccaaccggtctgcgcctgtgcagacgtagtcttcggcggmnnmnnmnnactctggtggtttgtg |
| | (SEQ ID NO.: 57) |
| WPP-mut-R2 | gtattccttggttttgaacccaaccggtctgcgcctgtgcagacgtagtcttcggmnnmnnmnnggcactctggtggtttgtg |
| | (SEQ ID NO.: 58) |
| WPP-mut-R3 | gtattccttggttttgaacccaaccggtctgcgcctgtgcagacgtagtcttmnnmnnmnnttgggcactctggtggtttgtg |
| | (SEQ ID NO.: 59) |
| WPP-mut-R4 | gtattccttggttttgaacccaaccggtctgcgcctgtgcagacgtagtmnnmnnmnnccattgggcactctggtggtttgtg |
| | (SEQ ID NO.: 60) |
| WPP-mut-R5 | gtattccttggttttgaacccaaccggtctgcgcctgtgcagacgtmnnmnnmnncggccattgggcactctggtggtttgtg |
| | (SEQ ID NO.: 61) |
| WPP-mut-R6 | gtattccttggttttgaacccaaccggtctgcgcctgtgcagamnnmnnmnncggcggccattgggcactctggtggtttgt |
| | g (SEQ ID NO.: 62) |
| WPP-mut-R7 | gtattccttggttttgaacccaaccggtctgcgcctgtgcmnnmnnmnncttcggcggccattgggcactctggtggtttgtg |
| | (SEQ ID NO.: 63) |
| WPP-mut-R8 | gtattccttggttttgaacccaaccggtctgcgcctgmnnmnnmnnagtcttcggcggccattgggcactctggtggtttgtg |
| | (SEQ ID NO.: 64) |
| WPP-mut-R9 | gtattccttggttttgaacccaaccggtctgcgcmnnmnnmnncgtagtcttcggcggccattgggcactctggtggtttgtg |
| | (SEQ ID NO.: 65) |
| WPP-mut-R10 | gtattccttggttttgaacccaaceggtctgcgcmnnmnnagacgtagtcttcggcggccamnnmnnactctggtggtttgt |
| | g (SEQ ID NO.: 66) |
| QRP-mut-R1 | gtattccttggttttgaacccaaccggtctgcgcctgtgccggctcacgcggaggcctmnnmnnmnnactctggtggtttgt |
| | g (SEQ ID NO.: 67) |
| QRP-mut-R2 | gtattccttggttttgaacccaaccggtctgcgcctgtgccggctcacgcggaggmnnmnnmnnggcactctggtggtttgt |
| | g (SEQ ID NO.: 68) |
| QRP-mut-R3 | gtattccttggttttgaacccaaccggtctgcgcctgtgccggctcacgcggmnnmnnmnnttgggcactctggtggtttgtg |
| | (SEQ ID NO.: 69) |
| QRP-mut-R4 | gtattccttggttttgaacccaaccggtctgcgcctgtgccggctcacgmnnmnnmnnctgttgggcactctggtggtttgtg |
| | (SEQ ID NO.: 70) |
| QRP-mut-R5 | gtattccttggttttgaacccaaccggtctgcgcctgtgccggctcmnnmnnmnncctctgttgggcactctggtggtttgtg |
| | (SEQ ID NO.: 71) |
| QRP-mut-R6 | gtattccttggttttgaacccaaccggtctgcgcctgtgccggmnnmnnmnnaggcctctgttgggcactctggtggtttgtg |
| | (SEQ ID NO.: 72) |
| QRP-mut-R7 | gtattccttggttttgaacccaaccggtctgcgcctgtgcmnnmnnmnncggaggcctctgttgggcactctggtggtttgtg |
| | (SEQ ID NO.: 73) |
| QRP-mut-R8 | gtattccttggttttgaacccaaccggtctgcgcctgmnnmnnmnnacgcggaggcctctgttgggcactctggtggtttgtg |
| | (SEQ ID NO.: 74) |
| QRP-mut-R9 | gtattccttggttttgaacccaaccggtctgcgcmnnmnnmnnctcacgcggaggcctctgttgggcactctggtggtttgtg |
| | (SEQ ID NO.: 75) |
| QRP-mut-R10 | gtattccttggttttgaacccaaccggtctgcgcmnnmnncggctcacgcggaggcctctgmnnmnnactctggtggtttg |
| | tg (SEQ ID NO.: 76) |
| Cap1-insertion-F | agaactcaaaatcagtccggaagtgc (SEQ ID NO.: 77) |
| Cap1-591-7MNN- | gcatgcacatctccggtcgcmnnmnnmnnmnnmnnmnnmnnagggtctgtgctgctgctctg (SEQ ID |
| R | NO.: 78) |
| Cap1-F | ccttttactgcctggaatatttcccttctc (SEQ ID NO.: 79) |
| Cap1-591i-R | gcatgcacatctccggtcgc (SEQ ID NO.: 80) |
| VNM-mut-R1 | gcatgcacatctccggtcgcaggcctagtatgcatattmnnmnnmnntgtgctgctgctctggaaattg (SEQ ID |
| | NO.: 81) |
| VNM-mut-R2 | gcatgcacatctccggtcgcaggcctagtatgcatmnnmnnmnngtctgtgctgctgctctggaaattg (SEQ ID |
| | NO.: 82) |
| VNM-mut-R3 | gcatgcacatctccggtcgcaggcctagtatgmnnmnnmnnagggtctgtgctgctgctctggaaattg (SEQ ID |
| | NO.: 83) |
| VNM-mut-R4 | gcatgcacatctccggtcgcaggcctagtmnnmnnmnnaacagggtctgtgctgctgctctggaaattg (SEQ ID |
| | NO.: 84) |
| VNM-mut-R5 | gcatgcacatctccggtcgcaggcctmnnmnnmnnattaacagggtctgtgctgctgctctggaaattg (SEQ ID |
| | NO.: 85) |
| VNM-mut-R6 | gcatgcacatctccggtcgcaggmnnmnnmnncatattaacagggtctgtgctgctgctctggaaattg (SEQ ID |
| | NO.: 86) |
| VNM-mut-R7 | gcatgcacatctccggtcgcmnnmnnmnnatgcatattaacagggtctgtgctgctgctctggaaattg (SEQ ID |
| | NO.: 87) |
| VNM-mut-R8 | gcatgcacatctccggtmnnmnnmnnagtatgcatattaacagggtctgtgctgctgctctggaaattg (SEQ ID |
| | NO.: 88) |
| VNM-mut-R9 | gcatgcacatctccmnnmnnmnncctagtatgcatattaacagggtctgtgctgctgctctggaaattg (SEQ ID |
| | NO.: 89) |
| VNM-mut-R10 | gcatgcacatctccmnnmnnaggcctagtatgcatattaacmnnmnntgtgctgctgctctggaaattg (SEQ ID |
| | NO.: 90) |

To identify AAV-cMG.WPP, AAV-cMG.QRP, and AAV-cMG.VNM variants that have enhanced performances, a AAV-cMG.WPP, AAV-cMG.QRP, and AAV-cMG.VNM capsid mutant libraries in which the inserted heptamer and the four flanking amino acids in the capsid were randomized was. Briefly, the library fragments were generated by ten separated PCR reactions using the XF and WPP-mut-R1-10 primers with the pCRII-9Cap-xE plasmid serving as the template, or using the XF and QRP-mut-R1-10 primers with the pCRII-9Cap-xE plasmid serving as the template, or using the Cap1-insertion-F and VNM-mut-R1-10 primers with the pCap1-T-mut serving as the template. Equal amounts of ten PCR products were mixed and assembled into the pAAV-CMV-mScarlet-ΔCap1/9-DIO-SV40pA plasmid using Gibson assembly. The resulted library was packaged into rAAVs as described above. For in vitro screening, the capsid mutant library rAAVs were applied to cultured mouse microglia as described above. For in vivo screening, the capsid mutant library rAAVs were injected bilaterally into the striatum (800 nL) and the midbrain (500 nL) of three Cx3cr1^{CreER} mice. Tamoxifen was injected (i.p., 10 mg/kg) for five consecutive days following virus injection. Mice were sacrificed ten days after virus injection. The brains were dissected, and the genomes of rAAVs that have successfully transduced cells in vivo were recovered using Trizol. The cap sequences in the Cre-recombined genomes were selectively amplified using the 9CapF and CDF primers. The candidates that were highly enriched were identified through NGS and individually tested. The enrichment score of a variant was calculated as follows:

$$Enrichment score = {Log}_{10} ( ( normalized read counts in screened sample ) \text{} / ( normalized read counts in the AAV library ) ) .$$

Immunohistochemistry. Cells were first washed in cold PBS and then fixed in 4% PFA for 10 min at room temperature. After washed again in PBS, cells were permeabilized in PBST and blocked in 2% BSA in PBST at room temperature for 20 min. Cells were then incubated with antibodies (anti-HA, 1:500, 11867423001, Roche) at room temperature for 2 hours. Cells were washed three times in PBST and were then incubated with fluorescent secondary antibodies (Goat anti-rat-Cy5, 1:1000, 112-175-143, Jackson ImmunoResearch) at room temperature for 1 hour.

Flow cytometry. Cultured mouse microglia were tested for B7H3-CAR expression using a two-step staining protocol: purified biotinylated human B7H3 ECD (11188-H27H-B-100, Sino Biological) primary stain followed by Streptavidin-AF647 (016-600-084, Jackson ImmunoResearch) secondary stain.

Bead-based phagocytosis assay. Strepavidin-coated polystyrene microparticles (5.0-5.9 μm diameter, Spherotech) were sterilized for 20 min in 70% isopropanol. Beads were spun down and resuspended in 0.1 M sodium bicarbonate buffer (pH 8.5) and labeled with 10 μM pHrodo SE (P36600, Thermo Scientific) for 30 min in the dark. Beads were spun down to remove free dye and resuspended in PBS. Biotinylated human B7H3 ECDs (11188-H27H-B-100, Sino Biological) were added to the beads at a concentration sufficient to occupy one quarter of the binding sites. Beads were incubated with protein for 1 h, washed and resuspended in PBS for use in experiments. Untransduced microglia, B7H3-CAR-MiAICD cells, and B7H3-CAR-Mi cells were plated at a density of 2.5×10⁴ cells per well in a 96-well plate and allowed to adhere. The media was aspirated and previously functionalized or blank beads in were added to obtain a 5:1 bead-to-cell ratio. Changes in fluorescence were monitored with Opera Phenix High Content Screening System (PerkinElmer).

FACS-based phagocytosis assay. 2.5×10⁵ untransduced microglia, B7H3-CAR-MiΔICD cells, B7H3-CAR-Mi cells, or CD19-CAR-Mi cells were co-cultured with 2.5×10⁵ mCherry-expressing GL261-B7H3-ECD cells in microglia culture medium for 4 h at 37° C. After co-culture, cells were harvested with Trypsin-EDTA (Gibco), stained with Anti-CD11b FITC (101206, BioLegend) and analyzed with FACS. The percent of mCherry⁺ events within the CD11b population was plotted as percentage phagocytosis.

Microscopy-based phagocytosis assay. GFP-expressing B7H3-CAR-Mi cells were were plated at 2.5×10⁴ per well in 96-well cell culture plate (6005550, PerkinElmer). 2.5×10⁴ mCherry-expressing U87 cells were added and co-cultured in microglia culture medium for 2 h at 37° C. After 2 h, tumor cells (nonadherent) were washed out. The plate was imaged for GFP and mCherry fluorescence, the cells were co-cultured and imaged every 2 min in 37° C. imaging chamber of Opera Phenix High Content Screening System (PerkinElmer) for 22 h.

Quantitative RT-PCR analysis. 2.5×10⁵ untransduced microglia (WT MG), 2.5×10⁵ B7H3-CAR-Mi cells (CAR-Mi), or 2.5×10⁵ B7H3-CAR-Mi cells co-cultured with 2.5×10⁵ GL261-B7H3-ECD cells (CAR-Mi+GBM) were placed in microglia culture medium in 12-well cell culture plate (Corning), cell culture medium was collected after 24 h. Primary microglia with or without 24 h exposure to 20 ng/ml IL-4 were seeded at 2.5×10⁵ per well in 12-well cell culture plate, replaced the medium with aforementioned collected cell culture medium, after another 24 h culture, total RNA was isolated with TRIzol. Reverse transcription was carried out using 5× All-In-One qPCR SuperMix (AE341-02, Transgen) and qPCR reactions were carried out by using the 2× Taq Pro Universal SYBR qPCR Master Mix (LIN B1260LBB, Vazyme) on CFX96 Real-Time System (Bio-Rad). Relative mRNA expression was calculated using the 2(−ΔΔCT) method. GAPDH was used as an internal control for samples. Primer sequences are summarized in Table 1.

Proinflammatory cytokine measurement. 2.5×10⁵ untransduced microglia (WT MG), 2.5×10⁵ B7H3-CAR-Mi cells (CAR-Mi), 2.5×10⁵ GL261-B7H3-ECD cells (GBM) or 2.5×10⁵ B7H3-CAR-Mi cells co-cultured with 2.5×10⁵ GL261-B7H3-ECD cells (CAR-Mi+GBM) were placed in 12-well cell culture plate (Corning), cell culture medium was collected after 12 h for proinflammatory cytokine measurement using IL-6 (EMC004.96.2, Neobioscience) and TNF (ADI-900-047, Enzo Life Sciences) ELISA kits.

Microglia isolation from adult mouse brain. We employed a cold-mechanical dissociation protocol as described previously⁵ with minor modifications. All procedures were performed on ice with cold buffers or in refrigerated centrifuge. Cx3cr1^{GFP} mice were deeply anesthetized and perfused. Brains were quickly removed and immersed in Dounce buffer (HBSS with HEPES+DNase+RNase inhibitor) and cut into smaller chunks. The tissue solution was quickly transferred to a 15 mL Dounce homogenizer and gently homogenized with a loose-fitting pestle for ˜10 times. The remaining tissue pieces were allowed to sediment and the supernatant containing cell suspensions were collected to a new tube. New Dounce buffer was added to the sediment tissue and the homogenization was repeated for another round. The collected cell solution was centrifuged, resuspended, and passed sequentially through 70-μm and 30-μm pre-wet cell strainers to remove debris. The cells were centrifuged once more and resuspended in 37% stock isotonic Percoll (SIP). A Percoll gradient of HBSS/30%/37% (cells)/70% was used to enrich microglia by centrifugating at 200 g for 20 mins with minimal acceleration and no brake. Cells in the interphase between 30% and 37% were carefully collected, washed, and resuspended in 0.04% BSA in Dulbecco's PBS.

Microglia replacement by microglia transplantation (mrMT). About 2×10⁴ brain microglia collected from adult Cx3cr1^{GFP} donor mice were stereotaxically microinjected into the lateral ventricle (AP: −0.58 mm, ML: ±1.25 mm, DV: −2 mm) of anesthetized Cx3cr1^{CreER}. Rosa26-LSL-DTA mice. After that, the recipient mice were administered tamoxifen (100 mg per kg of body weight, Sigma, T5648) dissolved in corn oil through intraperitoneal injection for 3 days to genetically ablate resident microglia. Mice were sacrificed two weeks after exogenous microglia transplantation, brains were dissected and sectioned after post-fixation and dehydration.

Stereotaxic intracranial tumor implantation. During stereotaxical microinjection, Cx3cr1^{CreER}: Rosa26-LSL-DTA mice were anesthetized with 2% isoflurane with oxygen. 3×10⁵ B7H3-CAR-Mi cells or untransduced microglia were stereotaxically microinjected into the lateral ventricle (AP: −0.58 mm, ML: ±1.25 mm, DV: −2 mm). Mice received tamoxifen administration for 3 consecutive days. Two weeks later, 2×10⁴ Firefly luciferase-expressing GL261-B7H3-ECD cells were stereotaxically microinjected into the hippocampus (AP: −1.5 mm, ML: 1.25 mm, DV: −2 mm) of mrMT mice. Bioluminescent imaging was performed every five days using an IVIS Spectrum (PerkinElmer).

Alternatively, 2×10⁴ firefly luciferase-expressing GL261-B7H3-ECD cells were stereotaxically microinjected into the hippocampus of Cx3cr1^{CreER}: Rosa26-LSL-DTA mice. The tumor growth was monitored by bioluminescent imaging. After tumor growth, 3×10⁵ B7H3-CAR-Mi cells or untransduced microglia were stereotaxically microinjected into the lateral ventricle. Mice received tamoxifen administration for 3 consecutive days. Bioluminescent imaging was performed every five days to track the changes of tumor progression.

To test CAR-Mi in wildtype mice, 2×10⁴ Firefly luciferase-expressing GL261-B7H3-ECD cells were stereotaxically microinjected into the hippocampus of C57BL/6N mice. The tumor growth was monitored by bioluminescent imaging. After tumor growth, mice were fed with food containing BLZ945 (2 g/kg), a small-molecule inhibitor of colony stimulating factor 1 receptor (Csf1r). One week later, we transplanted 3×10⁵ B7H3-CAR-Mi cells or untransduced microglia by icv injection and fed the mice with normal food. Bioluminescent imaging was performed every five days to track the changes of tumor progression.

### EXAMPLES

### Example 1. Screen of the Capsid Library from AAV9 in vitro.

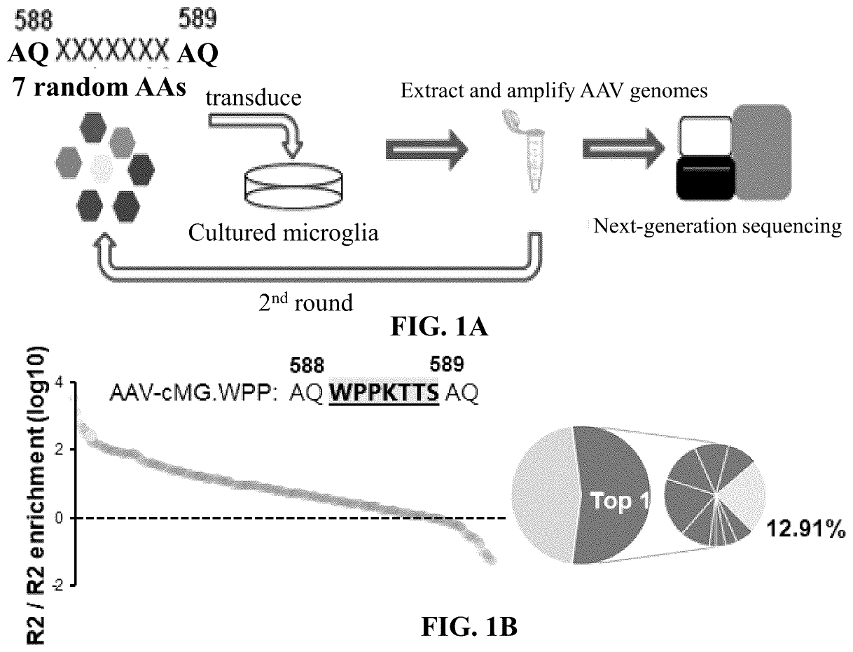

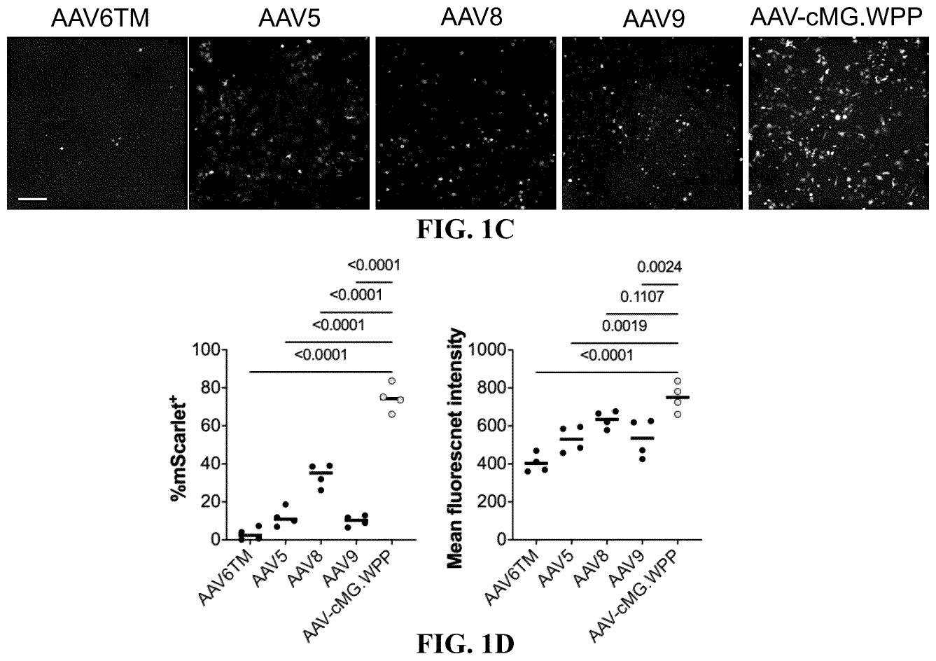

The wildtype AAV9 capsid was used as the starting point for generating a capsid library, in which each AAV9 capsid variant harbors a random seven-amino-acid insertion between amino acids 588 and 589 of the AAV9 VP1 protein (FIG. 1A). This library was packaged into rAAVs and screened in cultured mouse microglia for two consecutive rounds. The cultured mouse microglia were transduced with the capsid library rAAVs and the capsid variants that have successfully mediated transduction were recovered. Then, the recovered capsid variants were packaged into rAAVs and screened again in cultured mouse microglia. By next-generation sequencing, the capsid variants that were highly enriched after two rounds of screening were identified.

Two capsid variants, one harbors a “WPPKTTS” heptamer insertion (hereinafter referred to as AAV-cMG.WPP) and the other harbors a “QRPPREP” heptamer insertion (hereinafter referred to as AAV-cMG.QRP), showed significantly higher transduction of cultured microglia, as compared to the other candidates tested. The VP1 protein of AAV-cMG.WPP has an amino acid sequence as shown by SEQ ID NO.: 32, and the VP1 protein of AAV-cMG.QRP has an amino acid sequence as shown by SEQ ID NO.: 33. Then, single-stranded mScarlet reporter vectors were packaged into rAAVs using candidate capsid variants, respectively, and were transduced into cultured mouse microglia. The transduction abilities of the capsid variants were evaluated as compared to the parental AAV9 capsid, as well as three AAV capsids [AAV5, AAV8, and AAV6with Y731F/Y705F/T492V triple mutation (AAV6TM) 28] that have been reported to transduce cultured mouse microglia.

AAV-cMG.WPP was enriched over 170-fold and made up 12.91% of the total recovered variants in the second round of screening (FIG. 1B). Dramatically higher transduction rate was achieved by AAV-cMG.WPP (˜75%) as compared with that by the AAV5 (˜12%), AAV6TM (˜3%), AAV8 (˜34%), or AAV9 (˜10%) capsid. AAV-cMG.WPP also drives significantly stronger mScarlet expression than that by the AAV5, AAV6TM, AAV8, or AAV9 capsid (FIGS. 1C and 1D).

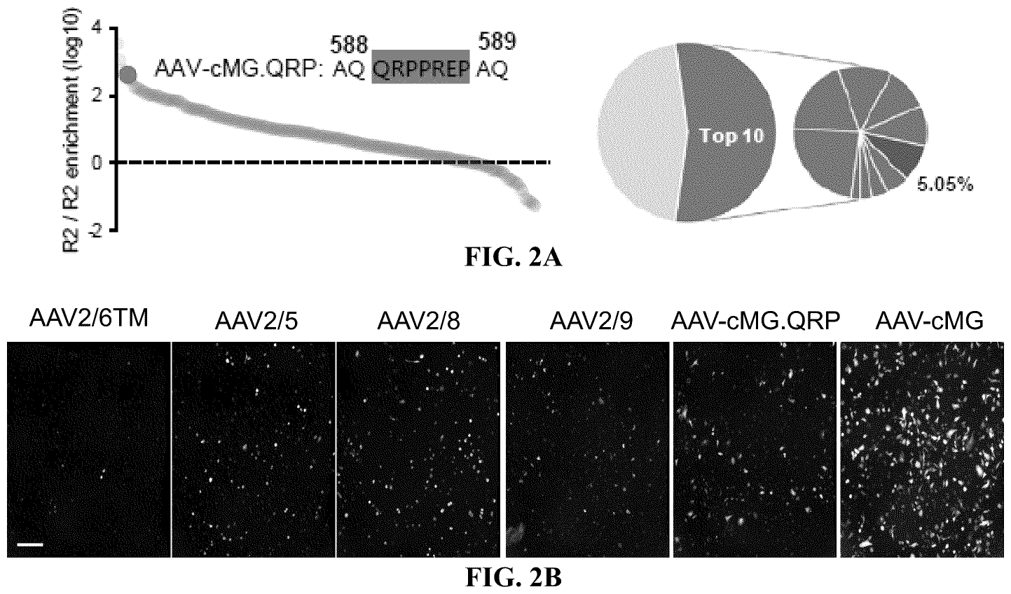

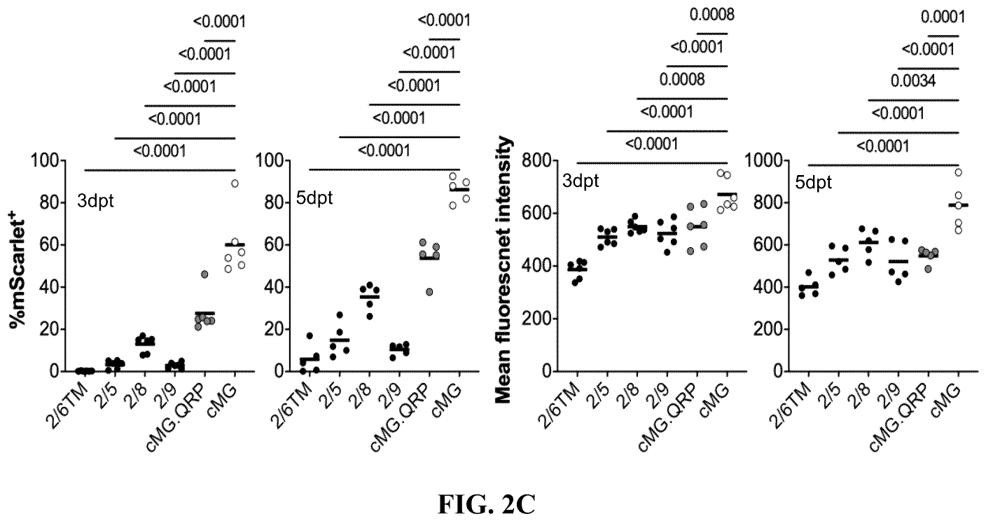

AAV-cMG.QRP was enriched ˜400-fold and made up 5.05% of the total recovered variants in the second round of screening (FIG. 2A). Significantly higher transduction rate and stronger mScarlet expression was achieved by AAV-cMG.QRP as compared with that by the AAV5, AAV6TM, AAV8, or AAV9 capsid (FIGS. 2B and 2C).

### Example 2. Further Screening of a Semi-Randomly Mutated Capsid Library from AAV-cMG. WPP for in vivo Transduction.

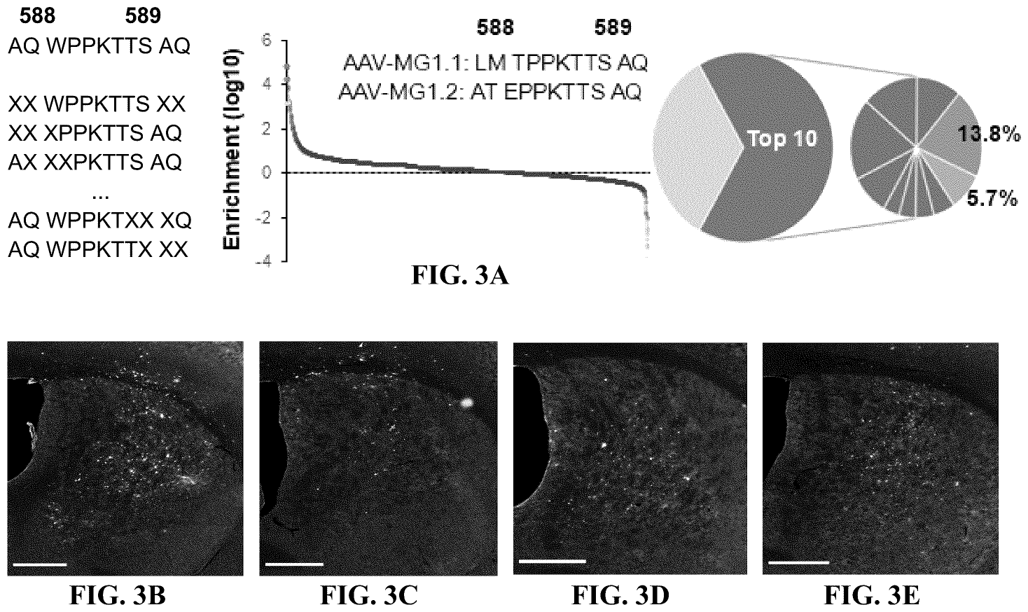

An additional capsid library was generated by semi-randomly mutating the inserted heptamer and the adjacent four amino acids in AAV-cMG.WPP. This new library was packaged into rAA Vs and screened in vivo by injecting the library rAAVs into the brains of Cx3cr1^{CreER} mice. The CREATE strategy was adopted to selectively recover capsid variants from Cre-recombined AAV genomes (i.e., genomes of rAAVs that have successfully transduced microglia in vivo). After two rounds of screening, two highly enriched capsid variants were identified (FIG. 3A), both of which contain mutations at amino acid positions 587-589 of AAV-cMG.WPP. The first variant comprises the amino acid sequence “LMT” at positions 587-589 and accounts for 13.8% of the total recovered variants. The second variant comprises the amino acid sequence “ATE” at positions 587-589 and account for 5.7% of the total recovered variants. These two AAV-cMG.WPP capsid variants were named as AAV-MG1.1 and AAV-MG1.2, respectively. The VP1 protein of AAV-MG1.1 has an amino acid sequence as shown by SEQ ID NO.: 35, and the VP1 protein of AAV-MG1.2 has an amino acid sequence as shown by SEQ ID NO.: 36.

Four additional AAV-cMG.WPP variants were also identified to be capable of transducing microglia in vivo and inducing strong and widespread mScarlet expression in the striatum of Cx3cr1^{CreER} mice. The first variant, AAV-MG.PTS, comprises the amino acid sequence “PTS” at positions 589-591 of AAV-cMG.WPP (FIG. 3B). The second variant, AAV-MG.LMV, comprises the amino acid sequence “LMV” at positions 589-591 of AAV-cMG.WPP (FIG. 3C). The third variant, AAV-MG.WTD, comprises the amino acid sequence “WTD” at positions 589-591 of AAV-cMG.WPP (FIG. 3D). The fourth variant, AAV-MG.VLS, comprises the amino acid sequence “VLS” at positions 588-590 of AAV-cMG.WPP (FIG. 3E). The VP1 protein of AAV-MG. PTS has an amino acid sequence as shown by SEQ ID NO.: 39. The VP1 protein of AAV-MG. LMV has an amino acid sequence as shown by SEQ ID NO.: 40. The VP1 protein of AAV-MG.WTD has an amino acid sequence as shown by SEQ ID NO.: 41. The VP1 protein of AAV-MG.VLS has an amino acid sequence as shown by SEQ ID NO.: 42.

### Example 3. Further Screen of a Semi-Randomly Mutated Capsid Library from AAV-cMG.QRP.

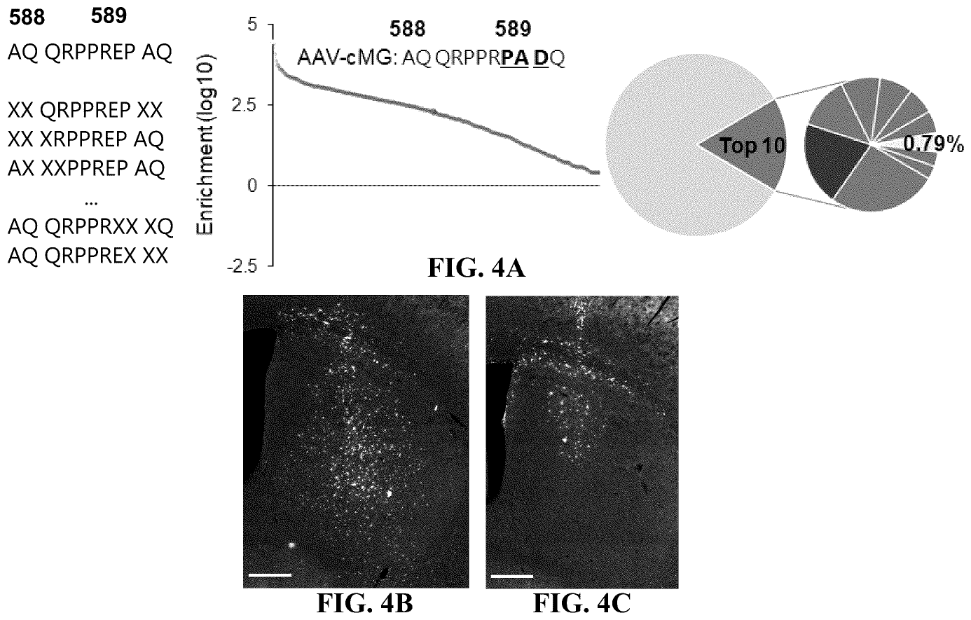

To further improve the transduction efficiency of AAV-cMG.QRP in cultured mouse microglia, an additional round of directed evolution was conducted on AAV-cMG.QRP. First, an additional capsid library was generated by semi-randomly mutating the inserted heptamer and the adjacent four amino acids in AAV-cMG.QRP. This new library was packaged into rAAVs and screened in cultured mouse microglia. After one round of screening, a highly enriched capsid variant was identified and contained mutations at amino acid positions 594-596 of AAV-cMG.QRP (FIG. 4A). The variant comprises the amino acid sequence “PAD” at positions 594-596 and accounts for 0.79% of the total recovered variants (FIG. 4A). This variant was named as AAV-cMG. Significantly higher transduction rate and stronger mScarlet expression was achieved by AAV-cMG compared with that by the AAV5, AAV6TM, AAV8, AAV9, or AAV-cMG.QRP capsid (FIG. 2B). The VP1 protein of AAV-cMG has an amino acid sequence as shown by SEQ ID NO.: 34.

The AAV-cMG.QRP mutant library was also screened in the brains of Cx3cr1^{CreER} mice. Two variants that are capable of transducing microglia in vivo were identified. The first variant, AAV-MG.TAF, comprises the amino acid sequence “TAF” at positions 589-591 of AAV-cMG.QRP (FIG. 4B). The second variant, AAV-MG.APA, comprises the amino acid sequence “APA” at positions 587-589 of AAV-cMG.QRP (FIG. 4C). The VP1 protein of AAV-MG.TAF has an amino acid sequence as shown by SEQ ID NO.: 37, and the VP1 protein of AAV-MG.APA has an amino acid sequence as shown by SEQ ID NO.: 38.

### Example 4. Screen of the Capsid Library from AAV1 in vitro.

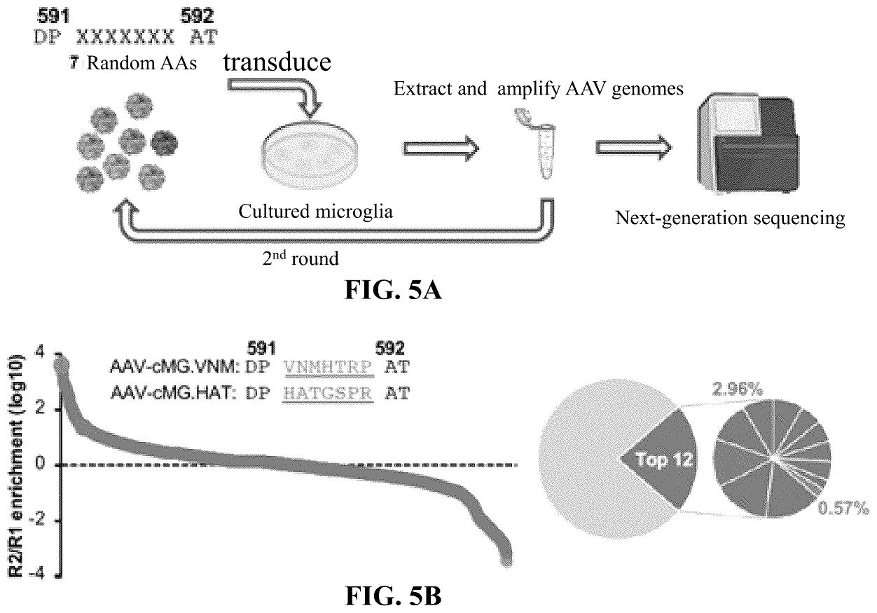

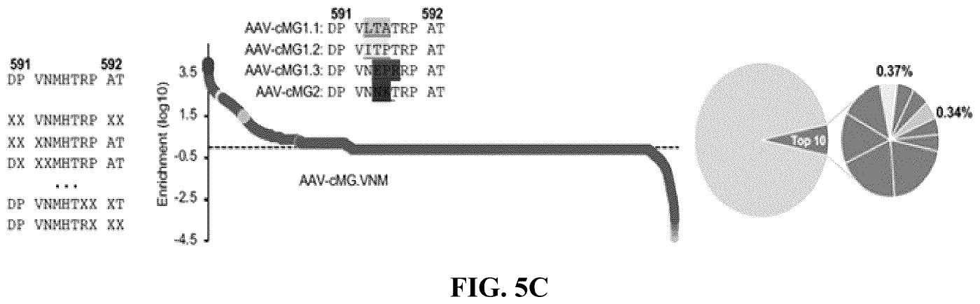

The wildtype AAV1 capsid was used as the starting point for generating a capsid library, in which each AAV1 capsid variant harbors a random seven-amino-acid insertion between amino acids 591 and 592 of the AAV1 VP1 protein (FIG. 5A). This insertion site is located at the protrusions of the capsid's threefold symmetry axis, which facilitates the interactions between inserted peptides and the membrane molecules on target cells. The library was packaged into rAAVs and screened in cultured mouse microglia for two consecutive rounds. The cultured mouse microglia were transduced with the capsid library rAAVs and the capsid variants that have successfully mediated transduction were recovered. Then, the recovered capsid variants were packaged into rAAVs and screened again in cultured mouse microglia. By next-generation sequencing, the capsid variants that were highly enriched after two rounds of screening were identified (FIG. 5B). Then, single-stranded mScarlet reporter vectors were packaged into rAAVs using candidate capsid variants and were transduced cultured mouse microglia with them individually. The transduction abilities and transgene expression levels of the capsid variants were evaluated against the parental AAV1, as well as the above AAV-cMG capsid.

Two capsid variants showed strong transgene expression level in cultured microglia among all candidates tested. The first variant harbors a “VNMHTRP” heptamer insertion (refer to as AAV-cMG.VNM afterwards; FIG. 5B). AAV-cMG.VNM was enriched over 3900-fold and made up 0.57% of the total recovered variants in the second round of screening. The second variant harbors a “HATGSPR” heptamer insertion (refer to as AAV-cMG.HAT afterwards; FIG. 5B). AAV-cMG.HAT was enriched over 20-fold and made up 2.96% of the total recovered variants in the second round of screening (FIG. 5B). The VP1 protein of AAV-cMG.VNM has an amino acid sequence as shown by SEQ ID NO: 25, and the VP1 protein of AAV-cMG.HAT has an amino acid sequence as shown by SEQ ID NO: 26.

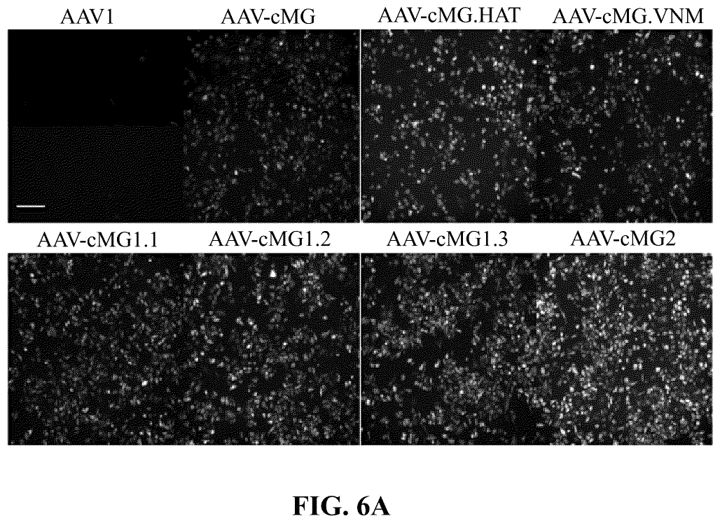

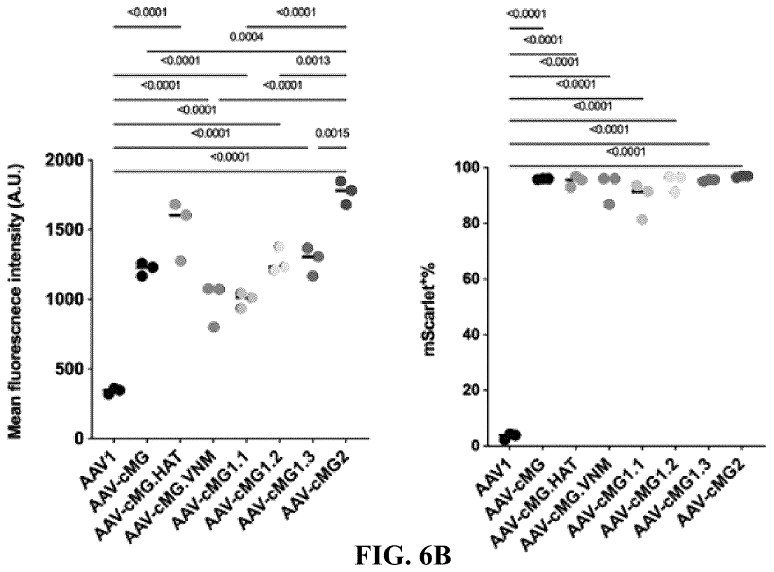

Both AAV-cMG.VNM and AAV-cMG.HAT drove significantly stronger mScarlet expression than that by the AAV1 or AAV-cMG capsid (FIGS. 6A and 6B). The transduction rate of AAV-cMG.VNM or AAV-cMG.HAT is comparable with that of AAV-cMG and is much higher than that of the parental AAV1. Notably, the AAV production yields of AAV-cMG.HAT and AAV-cMG.VNM are significantly higher than that of AAV-cMG (FIGS. 6A and 6B).

### Example 5. Further Screening of a Semi-Randomly Mutated Capsid Library from AAV-cMG.VNM.

To obtain variants that mediate even higher in vitro microglial transduction, the evolution of AAV-cMG.VNM was further conducted. A new capsid library was generated by semi-randomly mutating the inserted heptamer and the adjacent four amino acids in AAV-cMG.VNM (FIG. 5C). This library was packaged into rAAVs and screened in cultured mouse microglia.

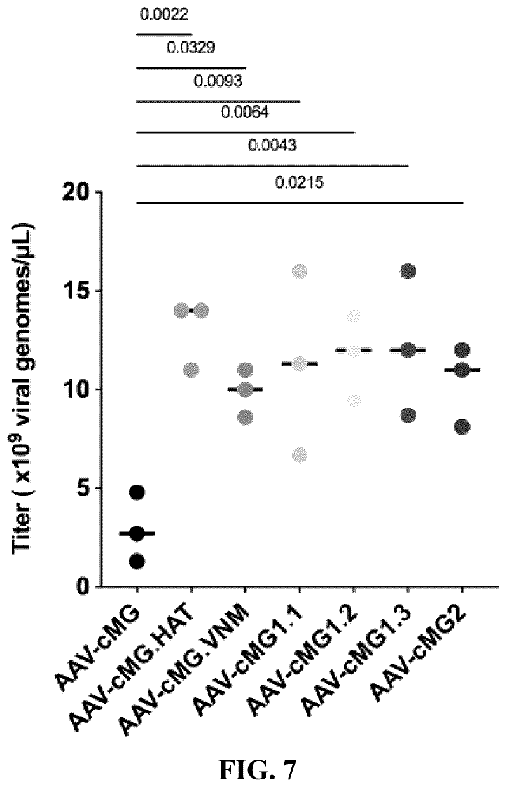

Four AAV-cMG.VNM variants that showed significantly enhanced transgene expression level compared with that by AAV-cMG were identified (FIG. 5C). The first variant harbors a “VLTATRP” heptamer insertion (refer to as AAV-cMG1.1 afterwards). AAV-cMG1.1 was enriched over 34-fold and made up 0.34% of the total recovered variants in the second round of screening. The second variant harbors a “VITPTRP” heptamer insertion (refer to as AAV-cMG1.2 afterwards). AAV-cMG1.2 was enriched over 260-fold and made up 0.37% of the total recovered variants in the second round of screening. The third variant harbors a “VNEPRRP” heptamer insertion (refer to as AAV-cMG1.3 afterwards). AAV-cMG1.3 was enriched over 6200-fold and made up 0.08% of the total recovered variants in the second round of screening. The fourth variant harbors a “VNNKTRP” heptamer insertion (refer to as AAV-cMG2 afterwards). AAV-cMG2 was enriched over 9900-fold and made up 0.13% of the total recovered variants in the second round of screening. The transduction rate of these AAV-cMG.VNM variants is comparable with that of AAV-cMG and is again much higher than that of the parental AAV1 (FIG. 6B). Also, the AAV production yields of AAV-cMG1.1, AAV-cMG1.2, AAV-cMG1.3, and AAV-cMG2 are significantly higher than that of AAV-cMG (FIG. 7).

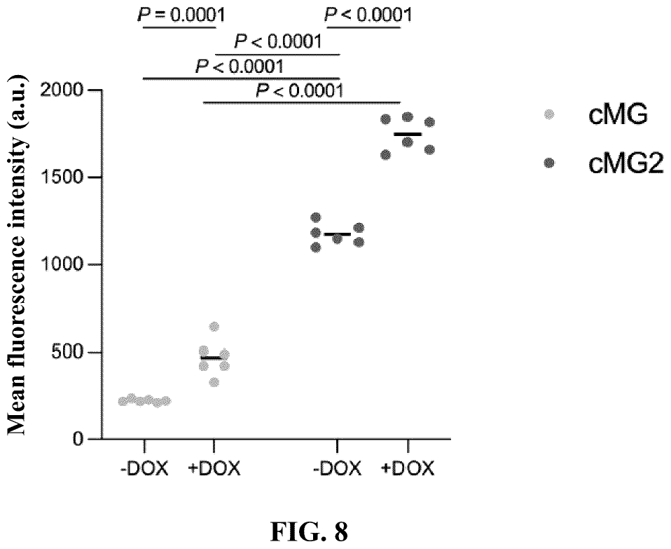

Previous studies have established that inhibiting topoisomerases and proteasomes using small-molecule drugs facilitates rAAV transduction, both in vitro and in vivo. Therefore, it was examined whether this approach may further enhance the microglial transduction efficiency of AAV-cMGs. An FDA-approved topoisomerase inhibitor doxorubicin, which has been shown to increase rAAV expression level in neurons in vivo, was used for this examination. For both AAV-cMG and AAV-cMG2, doxorubicin significantly enhanced the mScarlet expression level in cultured microglia (FIG. 8).

The VP1 protein of AAV-cMG1.1 has an amino acid sequence as shown by SEQ ID NO: 27, the VP1 protein of AAV-cMG1.2 has an amino acid sequence as shown by SEQ ID NO: 28, the VP1 protein of AAV-cMG1.3 has an amino acid sequence as shown by SEQ ID NO: 29, and the VP1 protein of AAV-cMG2 has an amino acid sequence as shown by SEQ ID NO: 30.

### Example 6. Construction of rAAV Vector for Delivering B7H3-CAR.

A single-stranded vector expressing the B7H3 mAb-CAR and a GFP reporter was packaged into rAAV-cMG2, resulting in AAV-cMG2-B7H3-CAR (FIG. 9A). Then, the resulted AAV-cMG2-B7H3-CAR transduced cultured mouse microglia. Strong GFP expression and correct membrane trafficking of B7H3-CAR were observed (FIG. 9B). When the biotinylated extracellular domain (ECD) of B7H3 to the medium, microglia that express B7H3-CAR (B7H3-CAR-Mi) efficiently bound the B7H3 ECDs, demonstrating that the CARs were functional. The upgraded AAV-cMG2 indeed drove stronger B7H3-CARs in microglia compared with the original AAV-cMG (FIG. 9C). Furthermore, doxorubicin, a topoisomerase inhibitor, could dramatically enhance the CAR expression driven by AAV-cMG2 (FIG. 9D).

### Example 7. Examination of Safety of AAV-cMG2-B7H3-CAR for Microglia.

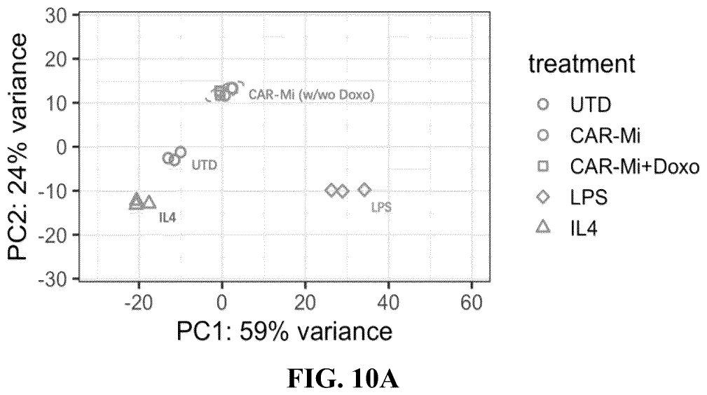

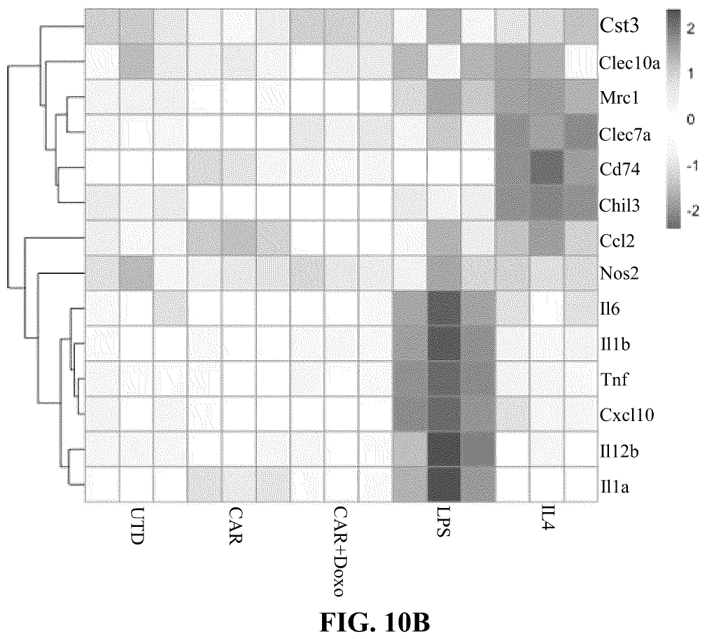

To examine whether AAV-cMG2-mediated B7H3-CAR expression and/or doxorubicin treatment trigger microglia phenotype changes, a principal component analysis was performed to obtain transcriptomes data from five different samples: control untransduced, endotoxin lipopolysaccharide (LPS)-treated, interleukin-4 (IL4)-treated, AAV-cMG2-B7H3-CAR-transduced, and doxorubicin-treated AAV-cMG2-B7H3-CAR-transduced cultured mouse microglia. Both AAV-cMG2-B7H3-CAR-transduced and doxorubicin-treated AAV-cMG2-B7H3-CAR-transduced microglia clustered towards control untransduced microglia, and away from LPS-treated or interleukin-4-treated microglia (FIG. 10A). Differential gene expression analysis also indicated that AAV-cMG2 transduction, B7H3-CAR expression, or doxorubicin treatment did not induce the proinflammatory pathways in cultured microglia (FIG. 10B). Thus, these results demonstrate 1) the utility of AAV-cMG2 to drive efficient CAR expression in microglia and 2) the safety of CAR expression in microglia without inducing microglial activation.

### Example 8. Target-Specific Phagocytosis of CAR-Modified Microglia.

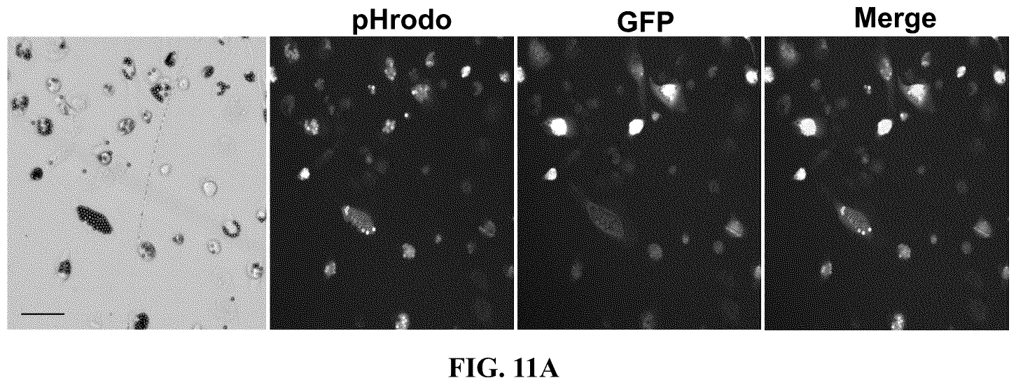

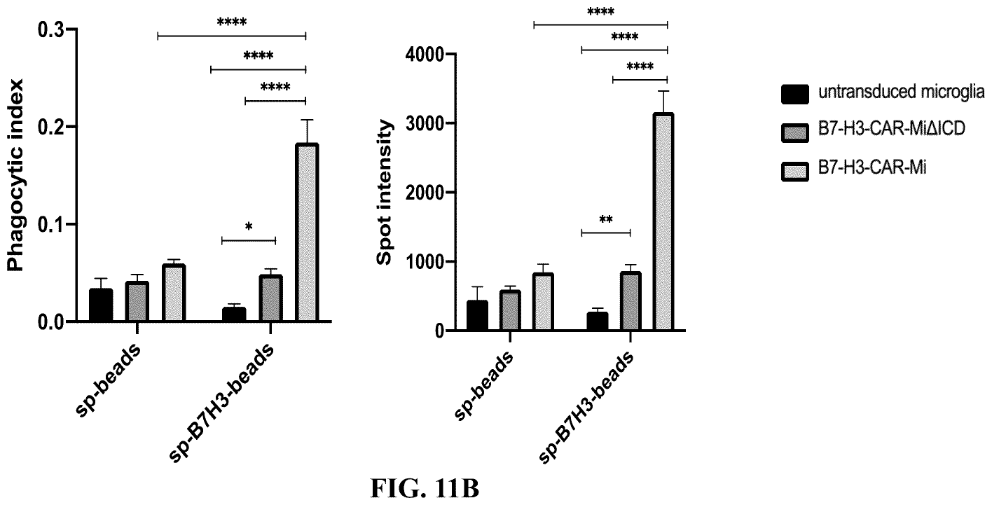

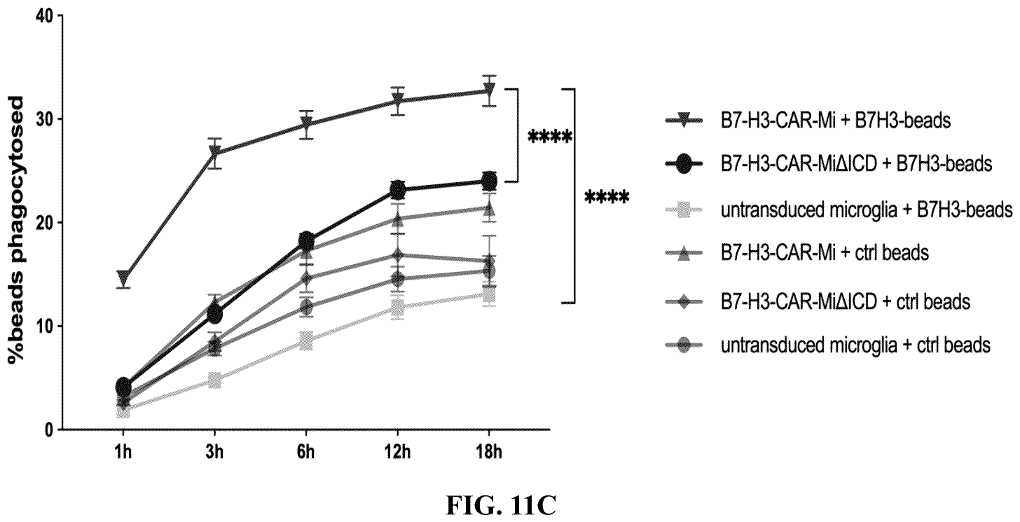

In this example, it examined whether CARs could direct microglia to perform target-specific phagocytosis. A pH-sensitive dye, pHrodo, was used to track microglial phagocytosis. Microsphere beads were modified with pHrodo and with purified human B7H3 ECDs (sp-B7H3-beads). Microsphere beads without human B7H3 ECDs (sp-beads) were used as control. Then, the sp-B7H3-beads or sp-beads were added to the medium of untransduced microglia, microglia that expressed B7H3-CAR without functional intracellular a domain (B7H3-CAR-MiAICDs) and B7H3-CAR-Mis. B7H3-CAR-Mis rigorously phagocytosed sp-B7H3 beads (FIG. 11A). The CAR-mediated enhancement in phagocytosis is target-specific, as B7H3-CAR-Mis did not mediate stronger phagocytosis of sp beads compared with untransduced microglia (FIGS. 11B and 11C). Also, B7H3-CAR-MiAICDs showed significantly weaker phagocytosis ability towards sp-B7H3 beads as compared with that of B7H3-CAR-Mi, demonstrating that CARs require a functional ICD to have activity (FIGS. 11B and 11C).

### Example 9. Specific Recognition and Phagocytosis of Tumor cells by CAR-Mi (hB7H3) Microglia

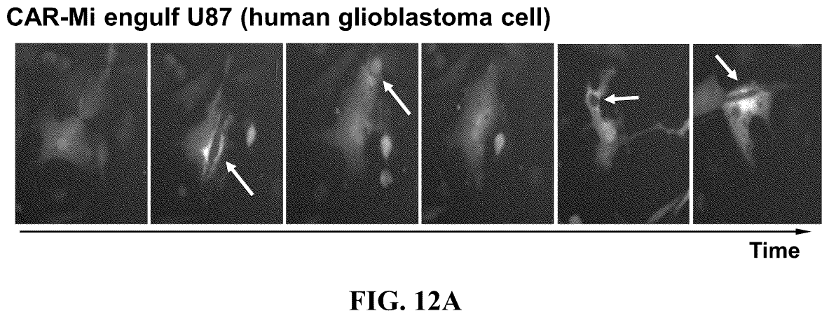

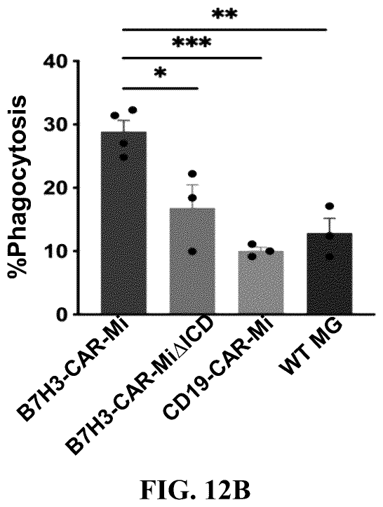

In this example, the capability of B7H3-CAR microglia (B7H3-CAR-Mi) was explored to attack cells that express target molecules. The B7H3-CAR microglia were co-cultured with U87 cells, an immortalized human glioma cell line that highly expresses B7H3. B7H3-CAR microglia were mobilized when they came into contact with U87 cells, leading to robust phagocytosis that remained constant over time (FIG. 12A). Another cell line was used to substantiate this finding. Human B7H3 ECDs were displayed on GL261 cells, an immortalized mouse glioma cell line that does not endogenously express B7H3. We then co-cultured B7H3 ECDs-expressing GL261 cells (GL261-B7H3-ECD) with untransduced microglia, B7H3-CAR-MiAICDs, B7H3-CAR-Mi or microglia that expressed CD19-targeting CARs (CD19-CAR-Mi). Fluorescence-activated cell sorting (FACS) was used to quantify the phagocytosis activities (FIG. 12B). Only B7H3-CAR-Mi mediated enhanced phagocytosis of target cells, again confirming the phagocytosis of CAR-Mi is target-specific and require full-length functional CARs.

### Example 10. CAR-Mediated Microglial Activation

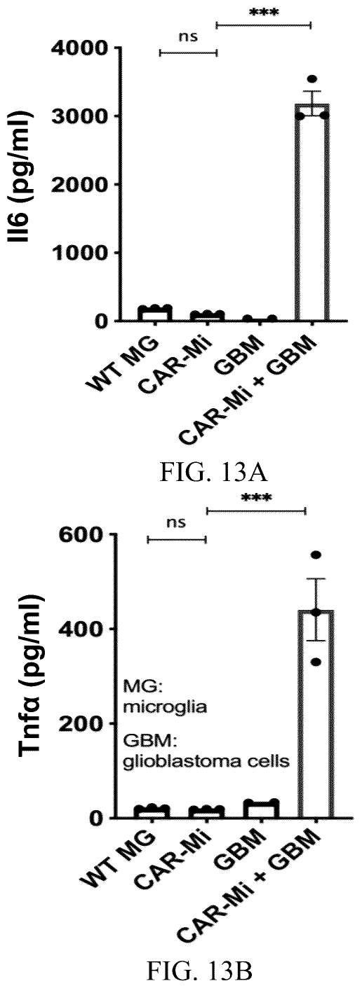

In this example, CAR-Mi was co-cultured with GL261-B7H3-ECD cells for 12 hours. Then, the culture medium was collected and detected for the levels of IL6 and TNF-α by means of ELISA. As compared to untransdued microglia (UTD), CAR microglia without tumor cells (CAR-Mi) and GL261-B7H3-ECD cells (GMB), the levels of both IL6 and TNF-α were significantly higher in the medium of CAR microglia co-culturing with tumor cells (CAR-Mi+GMB) (FIGS. 13A and 13B). These results demonstrate that CAR microglia can be activated by target tumor cells, and be induced to release pro-inflammatory cytokines.

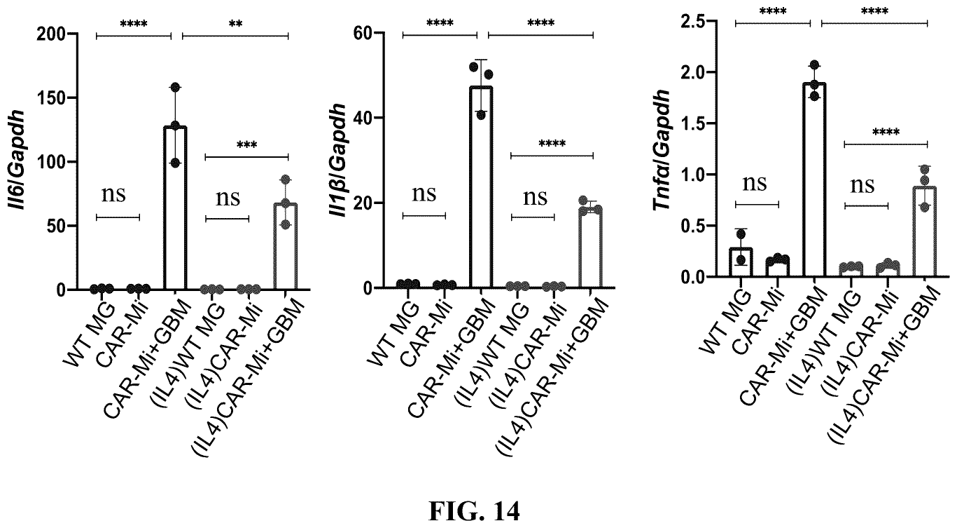

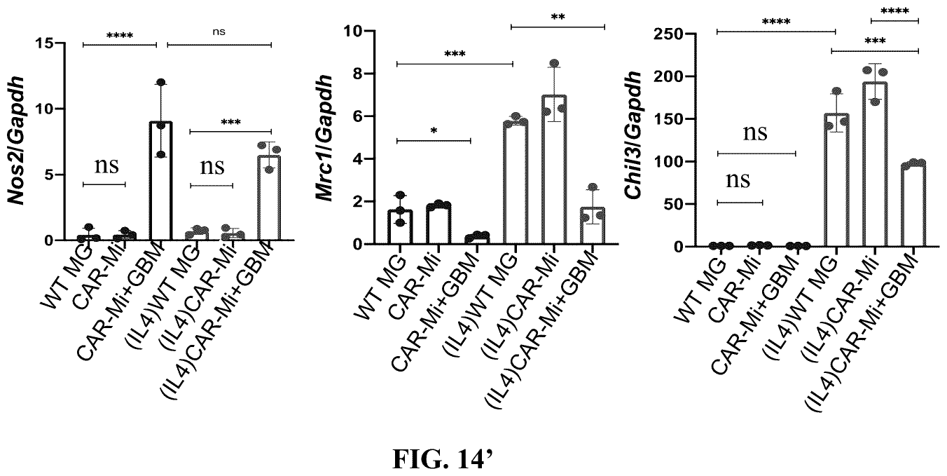

For both homeostatic microglia or IL4-treated (anti-inflammatory) microglia, the B7H3-CAR-Mis and GL261-B7H3-ECD co-culture medium induced the up-regulation of pro-inflammatory marker genes and the down-regulation of anti-inflammatory marker genes (FIG. 14). These results show that CAR-Mi can perform target-specific phagocytosis, release pro-inflammatory cytokines, and activate by-stander microglia (i.e., endogenous microglia that do not express CAR) upon target cell recognition.

### Example 11. Viability and Activity of Cultured Microglia after Transplanting into the Brains of Mice

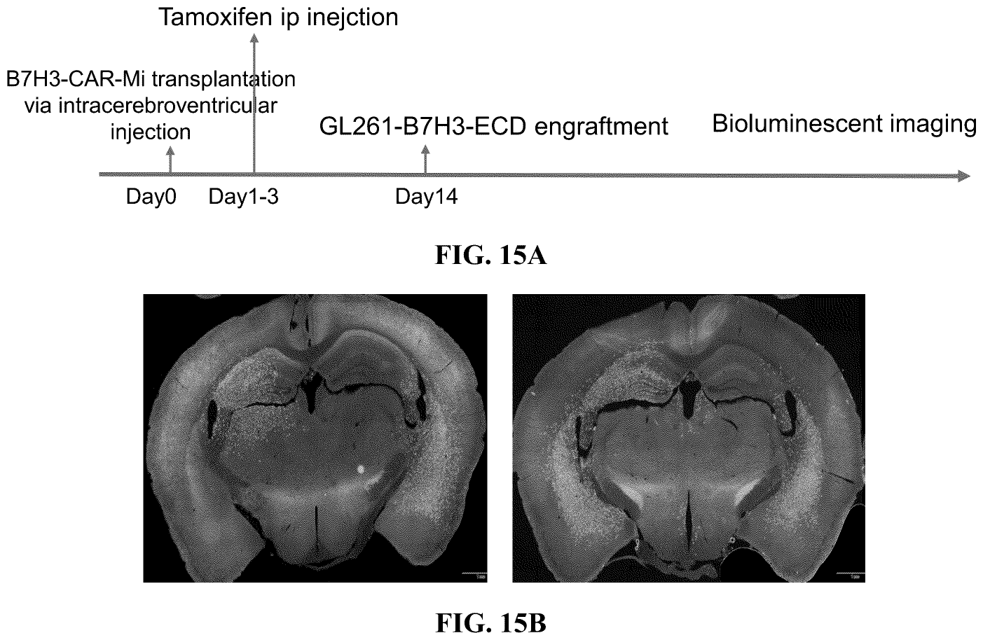

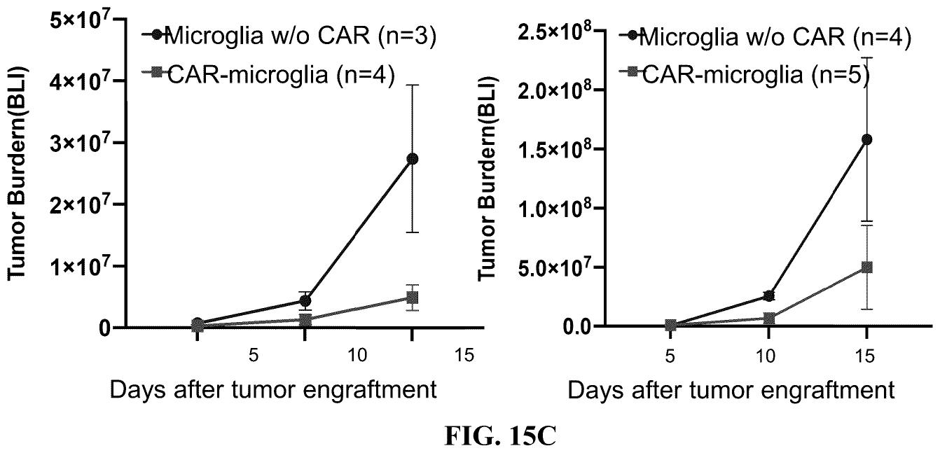

In this example, the performance of CAR-Mi was investigated in vivo. To facilitate the transplantation of CAR-Mi into the brain, endogenous microglia were depleted by means of a genetic approach. The Cre-dependent Rosa26 diphtheria toxin A (DTA) mice (Rosa26-LSL-DTA) were crossed with Cx3cr1^{CreER} mice which selectively express the tamoxifen-inducible Cre recombinase in microglia. Tamoxifen was intraperitoneal (ip) injected and induced DTA expression in microglia, resulting in subsequent cell death. B7H3-CAR-Mi were transplanted into the brains of Cx3cr1^{CreER}: Rosa26-LSL-DTA mice via intracerebroventricular (icv) injection after ip injecting tamoxifen for three consecutive days. Two weeks later, the mice were sacrificed to show whether GL261-B7H3-ECD cells were engrafted into the hippocampus (FIG. 15A).

With bioluminescent imaging, it can be seen that the GL261-B7H3-ECD cells were engrafted and highly expressed in the brain of the mice (FIG. 15B). In addition, B7H3-CAR-Mis significantly suppressed tumor growth in two independent cohorts of mice (FIG. 15C).

In addition, GL261-B7H3-ECD cells were first engrafted into the hippocampus of Cx3cr1^{CreER}: Rosa26-LSL-DTA mice. After tumor growth, B7H3-CAR-Mi were transplanted by icv injection, and the resident microglia were depleted via tamoxifen ip injection. B7H3-CAR-Mi-treated mice showed a marked reduction in tumor burden. A single infusion of B7H3-CAR-Mis led to a prolongation of overall survival. To further confirm these results, CAR-Mi were tested in wildtype mice. GL261-B7H3-ECD cells were engrafted into the hippocampus of wildtype mice. After tumor growth, the resident microglia were depleted by feeding mice with food containing BLZ945, a small-molecule inhibitor of colony stimulating factor 1 receptor (Csflr). One week later, B7H3-CAR-Mi were transplanted by icv injection and fed the mice with normal food. Again, it observed a significant reduction in tumor burden and prolonged survival in B7H3-CAR-Mi-treated mice. These results demonstrate the direct anti-tumor activity by CAR-Mi.

1. A recombinant adeno-associated virus (rAAV) vector, comprising a nucleic acid molecule encoding a chimeric antigen receptor (CAR) which specifically binds to a central nervous system (CNS) tumor cell, preferably a solid CNS tumor cell.

2. The rAAV vector according to claim 1, wherein the rAAV vector comprises a capsid protein, which has an inserted amino acid sequence of seven contiguous amino acids in a GH-loop of the capsid protein,
preferably, the capsid protein comprises an amino acid sequence selected from a group consisting of SEQ ID NO: 1, SEQ ID NO: 2, SEQ ID NO: 3, SEQ ID NO: 4, SEQ ID NO: 5 and SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 19, SEQ ID NO: 20, SEQ ID NO: 21, SEQ ID NO: 22, and SEQ ID NO: 23.

3. The rAAV vector according to claim 2, wherein the rAAV comprises VP1 capsid protein having an amino acid sequence selected from a group consisting of SEQ ID NOs: 25-30 and SEQ ID NO: 32-42, or an amino acid sequence having at least 85%, 90%, 95, 98% or 99% sequence identity thereof.

4. The rAAV vector according to claim 1, wherein the CAR comprises, from N-terminus to C-terminus: an antigen-binding domain which specifically binds to the CNS tumor cell; a hinge domain; a transmembrane domain; and an intracellular signaling domain.

5. The rAAV vector according to claim 4, wherein the antigen-binding domain specifically binds to a biomarker of the CNS tumor selected from a group consisting of B7-H1, B7-H3, B7-H4, B7-H5, B7-H7, BT3.1, natural-killer 2 receptor; natural-killer group 2, member D receptor protein; CD19; CD48; CD133; carcinoembryonic antigen; epidermal growth factor receptor; epidermal growth factor receptor variant III; epithelial cellular adhesion molecule; mucin 1; epidermal growth factor receptor 2; interleukin 13 receptor α2; EPH Receptor A2; Disialoganglioside 2, GD3, mesothelin, Tn Ag, PSMA, TAG72, CD44v6, KIT, leguman, CD171, IL-11Ra, PSCA, MAD-CT-1, MAD-CT-2, VEGFR2, LewisY, CD24, PDGFR-beta, SSEA-4, folate receptor alpha, ERBBs, NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, Polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, GPR20, Ly6k, OR51E2, TARP, or GFRa4;
the transmembrane domain comprises a polypeptide selected from a group consisting of T-cell receptor (TCR) alpha chain, a TCR beta chain, a TCR zeta chain, CD3 epsilon, CD4, CD5,CD8, CD9, CD16, CD22, CD27, CD28, CD33, CD45, CD80, CD83, CD86, CD134, CD137, CD152, CD154, CD279, PD-1 and a combination of any thereof, and the TMD preferably comprises an amino acid sequence as shown by SEQ ID NO:44 or an amino acid sequence having at least 85%, 90%, 95%, 98% or 99% sequence identity thereof;
the intracellular signaling domain comprises a first intracellular signaling domain derived from the group consisting of 4-1BB, CD27, CD28, OX40, CD70, LFA-2, CD5, ICAM-1, LFA-1, DAPIO, DAP12, a co-stimulatory inducible T-cell costimulatory polypeptide sequence, and a combination of any thereof, and the first intracellular signaling domain preferably comprises comprise an amino acid sequence as shown by SEQ ID NO:45 or an amino acid sequence having at least 85%, 90%, 95%, 98% or 99% sequence identity thereof;
the intracellular signaling domain optionally further comprises a second intracellular signaling domain derived from of CD3 zeta, of FCGR3A and of NKG2D, and a combination of any thereof, and the second intracellular signaling domain preferably comprises an amino acid sequence as shown by SEQ ID NO: 46 or an amino acid sequence having at least 85%, 90%, 95%, 98% or 99% sequence identity thereof.

6. A modified cell comprising a chimeric antigen receptor (CAR) which specifically binds to a central nervous system (CNS) tumor cell, preferably a solid CNS tumor cell.

7. The modified cell according to claim 6, wherein the CAR comprises, from N-terminus to C-terminus: an antigen-binding domain which specifically binds to the CNS tumor cell; a hinge domain; a transmembrane domain; and an intracellular signaling domain.

8. The modified cell according to claim 7, wherein the antigen-binding domain specifically binds to a biomarker of the CNS tumor selected from a group consisting of B7-H1, B7-H3, B7-H4, B7-H5, B7-H7, BT3.1, natural-killer 2 receptor; natural-killer group 2, member D receptor protein; CD19; CD48; CD133; carcinoembryonic antigen; epidermal growth factor receptor; epidermal growth factor receptor variant III; epithelial cellular adhesion molecule; mucin 1; epidermal growth factor receptor 2; interleukin 13 receptor α2; EPH Receptor A2; Disialoganglioside 2, GD3, mesothelin, Tn Ag, PSMA, TAG72, CD44v6, KIT, leguman, CD171, IL-11Ra, PSCA, MAD-CT-1, MAD-CT-2, VEGFR2, LewisY, CD24, PDGFR-beta, SSEA-4, folate receptor alpha, ERBBs, NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, Polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, GPR20, Ly6k, OR51E2, TARP, or GFRa4;
the transmembrane domain comprises a polypeptide selected from a group consisting of T-cell receptor (TCR) alpha chain, a TCR beta chain, a TCR zeta chain, CD3 epsilon, CD4, CD5, CD8, CD9, CD16, CD22, CD27, CD28, CD33, CD45, CD80, CD83, CD86, CD134, CD137, CD152, CD154, CD279, PD-1 and a combination of any thereof;
the intracellular signaling domain comprises a first intracellular signaling domain derived from the group consisting of 4-1BB, CD27, CD28, OX40, CD70, LFA-2 (CD2), CD5, ICAM-1, LFA-1, DAPIO, DAP12, a co-stimulatory inducible T-cell costimulatory polypeptide sequence, and a combination of any thereof;
the intracellular signaling domain optionally further comprises a second intracellular signaling domain derived from of CD3 zeta, of FCGR3A and of NKG2D, and a combination of any thereof.

9. The modified cell according to claim 6, which is obtained by introducing a chimeric antigen receptor (CAR) into the cell by using a recombinant adeno-associated virus (rAAV) vector, preferably the modified cell is a modified microglia and/or astrocyte,
wherein, the rAAV vector comprises a nucleic acid molecule encoding the CAR which specifically binds to a central nervous system (CNS) tumor cell, preferably a solid CNS tumor cell.

10. A method for obtaining a modified cell comprising a chimeric antigen receptor (CAR) which specifically binds to a central nervous system (CNS) tumor cell, preferably a solid CNS tumor cell, comprising transducing a cell, preferably a microglia and/or astrocyte, with the rAAV vector as defined in claim 1.

11. A method for treating a CNS tumor, preferably a solid CNS tumor, comprising administering to a subject a therapeutically effective amount of the rAAV vector as defined in claim 1.

12. The method according to claim 11, wherein the solid CNS tumor comprises gliomas, glioneuronal tumors, neuronal tumors, choroid plexus tumors, embryonal tumors, pineal tumors, cranial and paraspinal nerve tumors, meningiomas, mesenchymal non-meningothelial tumors, melanocytic tumors, germ cell tumors, and metastatic brain tumors.

13. The method according to claim 11, wherein the rAAV vector or the modified cell is administered by intratumoral or paratumoral injection.

14. A method for treating a CNS tumor, preferably a solid CNS tumor, comprising administering to a subject a therapeutically effective amount of the modified cell as defined in claim 6.

15. The method according to claim 14, wherein the solid CNS tumor comprises gliomas, glioneuronal tumors, neuronal tumors, choroid plexus tumors, embryonal tumors, pineal tumors, cranial and paraspinal nerve tumors, meningiomas, mesenchymal non-meningothelial tumors, melanocytic tumors, germ cell tumors, and metastatic brain tumors.

16. The method according to claim 14, wherein the rAAV vector or the modified cell is administered by intratumoral or paratumoral injection.

17. The modified cell according to claim 9, wherein the rAAV vector comprises a capsid protein, which has an inserted amino acid sequence of seven contiguous amino acids in a GH-loop of the capsid protein,
preferably, the capsid protein comprises an amino acid sequence selected from a group consisting of SEQ ID NO: 1, SEQ ID NO: 2, SEQ ID NO: 3, SEQ ID NO: 4, SEQ ID NO: 5 and SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 19, SEQ ID NO: 20, SEQ ID NO: 21, SEQ ID NO: 22, and SEQ ID NO: 23.

18. The modified cell according to claim 9, wherein the rAAV comprises VP1 capsid protein having an amino acid sequence selected from a group consisting of SEQ ID NOs: 25-30 and SEQ ID NO: 32-42, or an amino acid sequence having at least 85%, 90%, 95, 98% or 99% sequence identity thereof.
